SEQUENCE LISTING (15 sequences)

SEQ ID NO: 1 - DNA, 2872 bp, Homo sapiens
aggcggcgcgcacggccggataggcgcgagggggccgcgtgaggcggtgccggcgttctggcccccaaagccggtctagcgcgccgggcgtcttccttacttccgctgccgccgccgccacatcccgggacccgacgggccgcggcgcggaggcctcggggcaaggtggggcgggcctcccgagctcccaggaccccgcgcgcttcgcccacaggcccggcgaagcccgacccgcgcggcgcccccagggccaggggaggagcctaaggacccggacgagcgccgctccagtaggtgacaagaggaaccaagaacctcagttcaggggaaacacagcaaggaaatgtgagccccaggctgcagaaggaagagtcagtgaatggctgcggtgtgacaacatgcaccaccagtggcttctgctggccgcatgcttttgggtgattttcatgttcatggtggctagcaagttcatcacgttgacctttaaagacccagatgtgtacagtgccaaacaggagtttctgttcctgacaaccatgccggaagtgaggaagttgccagaagagaagcacattcctgaggaactgaagccaactgggaaggagcttccagacagccagctcgttcagcccctggtctacatggagcgcctggaactcatcagaaacgtctgcagggatgatgccctgaagaatctctcgcacactcctgtctccaagtttgtcctggaccgaatatttgtctgtgacaagcacaagattcttttctgccagactcccaaagtgggcaacacccagtggaagaaagtgctgattgttctaaatggagcattttcttccattgaggagatccccgaaaacgtggtgcacgaccacgagaagaacggccttcctcggctctcttccttcagtgatgcagaaattcagaagcgattgaaaacatacttcaagttttttattgtaagagatcccttcgaaagacttatttctgcatttaaggataaatttgttcacaatccccggtttgagccttggtacaggcatgagattgctcctggcatcatcagaaaatacaggaggaaccggacagagacccgggggatccagtttgaagatttcgtgcgctacctcggcgatccgaaccacagatggctagaccttcagtttggggaccacatcattcactgggtgacgtatgtagagctctgtgctccctgtgagataatgtacagtgtgattggacaccacgagaccctggaggacgatgccccatacatcttaaaagaggctggcattgaccacctggtgtcatacccgactatccctccgggcattaccgtgtataacagaaccaaggtggagcactatttcctgggcatcagcaaacgagacatccgacgcctgtatgcccgtttcgaaggggactttaagctctttgggtaccagaaaccagactttttgctaaactaatgcataagacctatgaattcaaatatctttattagacctggggctaaccaggtgaagatctgagcccagaaatgacccttcctccaccacacccctcctttgaggacgcccggggtctcccacaggcctgtgagttgcctcggcatatgacgcagaaccccaactgttacaacttagtttggatgtaagatgctctgaggaccctgcccacacccctgcgtgcattaggatgtcgctggcctttgctcacctcagaggggagaaaaggctaaagatttgcagtttgacagcccagcagggaggaagcatcacacagcgttaggagccgtttccttcaggtgttaaggaaggggatgcccctgaggttctcctggctagtcagggtggcttcacccatcactggtgggttgcaggaacagcacccaggactctgaggagggacagagaagcaagggggctgctgaaatcgcagagacttttgcagcatcagatctgaggagtaaaacggcacctctggccttcatcttggtgctgcgacaattgtggaggcaaagcattctttctgtgactattttgttcctgtagacagtcagcgatggccagagggtggtgtggtgtccaggggtccatctttccagaatccatgcctgtgtaatgctggtccatgcttctgaacctgtgtctgccaagcgcctatttcattcagcacaagacatacgattttagaaggtgaggggaggggaggctttttctacctgagaaggggagtgtctttgagggccttaaaaggaccatggcccaggaatgggggcgctggttgggcttggagctcaggctgctgtggatcccggcgcatcagttctgacttgccttacctgggtggacagcagtgaatctccacctgtcttctccagggagctcccatgttggggctgaagacgagcaggggcaacctgccagcatcacagaattcagtgtagtttatacatttcgattcctttcatctcagcaaaatgggcactgccagagccatttctgatcacaccaccatcctggaccatgtgactggaaggtgggtaaccaagttcaccagcaataaaacccagcgcccaggtagcctccagcagtgcggcttcctggcaacaaggtaggccctggtgcagggcaagccgcagcgaccatttcagataccgtccacagccaggaccgctgagaactgggacagtttcctgggatgagtgccagcctgagcctgcatggtgccgccgagcccggggtggaggagggagccaggcttcgcttcaaggcggcctctaccttttctcagaatggtttcctgattgtgtcaatgtgaaagttaaataaaatttatgtgccaaacctgaaaaaaaaaaaaaaaaaaa

SEQ ID NO: 2 - protein, 356 aa, Homo sapiens
MHHQWLLLAACFWVIFMFMVASKFITLTFKDPDVYSAKQEFLFLTTMPEVRKLPEEKHIPEELKPTGKELPDSQLVQPLVYMERLELIRNVCRDDALKNLSHTPVSKFVLDRIFVCDKHKILFCQTPKVGNTQWKKVLIVLNGAFSSIEEIPENVVHDHEKNGLPRLSSFSDAEIQKRLKTYFKFFIVRDPFERLISAFKDKFVHNPRFEPWYRHEIAPGIIRKYRRNRTETRGIQFEDFVRYLGDPNHRWLDLQFGDHIIHWVTYVELCAPCEIMYSVIGHHETLEDDAPYILKEAGIDHLVSYPTIPPGITVYNRTKVEHYFLGISKRDIRRLYARFEGDFKLFGYQKPDFLLN

SEQ ID NO: 3 - DNA, 1925 bp, Homo sapiens
ctttctttccttgctttgggatcttgctgctggatccggagaggttctgagaagacaagagcaagggactgagagcaggcttccgctgcggcgcgcgaacacagccgggacacaacccccagcgtctccacccgctcctcgccaccccggcgggaatgtgaggaaggaaagcccccagcgccgccgcccgccctcgaaggcgtcccagagagcgtcctgggggcccgcggctggagcccttgtgcccgcagcaccgccggactggagcggcgaggcgcaccgggtgccgcttctcggcttccactcttcagaaagagcgcggtggggatcagcgcctttcccgcactcggcacaactccgggaccggcggcgcgcggctggaccgagtcccgcttcccgccagctcacctggagtcgggggcagcccctgcccgcccgcctgcaccccttgtcgctctagcttgcgcgaacctgccgctcctccacgcccaggtagtgagccccgcggctccaggtctctgcagcgccctcggccccatggacagcgcacccatcaccactccctaagtgctggcgccgccgctgtccaagctgcgcactggggtccctcggctcgcccctctctggggtgtccgagaggccagggagcgtgcaccatgaagtccgcgcttttcacccgcttctttatcctcctgccctggatcctaattgtcatcatcatgctcgacgtggacacgcgcaggccagtgcccccgctcaccccgcgcccctacttctctccctacgcggtgggccgcgggggcgcccgactcccgctccgcaggggcggcccggctcacgggacccaaaagcgcaaccagtctcggccgcagccacagccggagccgcagctgcccaccatctatgccatcacgcccacctacagccgcccggtgcagaaagcggagctgacccgcctggccaacacgttccgccaggtggcgcagctgcactggatcctggtggaggacgcggcggcgcgcagcgagctggtgagccgcttcctggcgcgggccgggctgcccagcactcacctgcacgtgcccacgccgcggcgctacaagcggcccgggctgccgcgcgccactgagcagcgcaacgcgggcctcgcctggctgcgccagaggcaccagcaccagcgcgcgcagcccggcgtgctcttcttcgctgacgacgacaacacctatagtctggagctcttccaggagatgcgaaccacccgcaaggtctccgtctggcctgtgggcctggttggtgggcggcgctacgaacgtccgctggtggaaaacggcaaagttgttggctggtacaccggctggagagcagacaggccttttgccatcgacatggcaggatttgctgtaagtcttcaagtcattttgtccaatccaaaagctgtatttaagcgtcgtggatcccagccagggatgcaagaatctgactttctcaaacagataacaacagtcgaagaactggaaccgaaagcaaataactgcactaaggttctcgtgtggcacactcggacagagaaggttaatctagccaacgagccaaagtaccacctggacacagtgaaaattgaggtataaattgaagcagcaactggtgcagtttgtccagccagtggatccatatggaagaggatgtttggagtttaggctacagagcattcaggtattgtttgttttacttcagtacagcagcctttcttgtcatctgatggacatctgtttaaatggagcttgtcagttaacataagctaattggatggttggtacaaaatgtatgttttgtcttcatttgttctgcatgttttctctacaacaactaaattggaagatttttttgtacagtgccgatactgcaagataccactcttgagtata

SEQ ID NO: 4 - protein, 323 aa, Homo sapiens
MKSALFTRFFILLPWILIVIIMLDVDTRRPVPPLTPRPYFSPYAVGRGGARLPLRRGGPAHGTQKRNQSRPQPQPEPQLPTIYAITPTYSRPVQKAELTRLANTFRQVAQLHWILVEDAAARSELVSRFLARAGLPSTHLHVPTPRRYKRPGLPRATEQRNAGLAWLRQRHQHQRAQPGVLFFADDDNTYSLELFQEMRTTRKVSVWPVGLVGGRRYERPLVENGKVVGWYTGWRADRPFAIDMAGFAVSLQVILSNPKAVFKRRGSQPGMQESDFLKQITTVEELEPKANNCTKVLVWHTRTEKVNLANEPKYHLDTVKIEV

SEQ ID NO: 5 - DNA, 3534 bp, Homo sapiens
ggagggagccgcacgcggcccagggcagcggtctaggggcgccggggccggggcgtaggggccgttgcccgcgatggaccgcaccggagacgctccggactcgtcgccgcaggtgtccaccccccagggttcctgacccctgcccctggacagcgaccccttctcagactccagttgggccggactctccaaacctgcttccgcaatgggtgggttgtgagtgctggtaatgaggagccgtgggtgcagccagccttggagatgccgaagagacgggacatcctagcgatcgtcctcatcgtgctgccctggactctgctcatcactgtctggcaccagagcaccctcgcacccctgctcgcggtacataaggatgagggcagtgacccccgacgcgaaacgccgcccggcgccgaccccagggagtactgcacgtctgaccgcgacatcgtggaggtggtgcgcaccgagtacgtgtacacgcggcccccgccatggtccgacacgctgcccaccatccacgtggtgacgcccacctacagccgcccggtgcagaaggccgagctgacgcgcatggccaacacgctgctgcacgtgcccaacctccactggctggtggtggaggatgcgccgcgccggacgccgctgaccgcgcgcctgctgcgcgacaccggcctcaactacacgcacctgcacgtggagacgccccgcaactacaagctgcgcggagacgcccgcgacccacgcatcccgcggggcaccatgcagcgcaacctggccctgcgctggctgcgcgagaccttcccgcgcaactccagccagcctggcgtggtctacttcgccgacgacgacaacacctacagcctggagctcttcgaagagatgcgcagcaccaggagggtgtccgtgtggcccgtcgccttcgtgggtggcctgcggtacgaggccccacgggtgaacggggcagggaaggtggtcggctggaagacggtgtttgacccccaccggccatttgcaatagacatggctggatttgccgtcaacctgcggctcattctgcagcgaagccaggcctacttcaagctgcgaggtgtgaagggaggctaccaggaaagcagcctccttcgagaacttgtcaccctcaacgacctggagcccaaggcagccaactgcaccaagatcctggtgtggcacacacggacagagaagccagtgctggtgaatgagggcaagaagggcttcactgacccctcggtggagatctgagcctcaggatgcaggagcctcctcctcagaccctgttcttggccttccatcctctccccacggctgatggtccctccaaggccgactcctaaggaatcaccatcaccctcctttctattctgggggcttctgagagagcccagcctgatgccagaacaaaggacagagaatttaagcacagaaatcccagacctgttgttctctccatccagcgtgaccagggcccgagagacctgatggccagggtggggtgtccagcaccagccaagctggtgctccagcgcacctccccagagctccccgcactgacggggctgcaggagcaggtgcagtgggcgcccacactggccctgcagtgatgcagggcgggagggagataagaagaccccgcagtcaagtggagcatggccctccctggctccctgtccctgggctcagcacgaccacacaggacacccagccagggaattctgaagaccagagagcagcccacgggcatcacgagcgctctgctcctctcctgggcccctgctcttcccgagagctgcccccaaatcagacatacctctgtggctctcctctggttcacgtttacagagcataaggctgtcttggatcccaacaggcacccagccctgcatggggggagcctgggcctaataggcaccccctgtacctcaggctgtggcgggagcagagtccccccctccggcccctcttcctttaccccttctcctccagcagtggcaaaggggtaggctctagagccagcacaggtcactgcctgacctggactaagaaccccacggccccactgtccacacactgcctccccaccgcccacctcggctgctaggcccctcgcctggactggactggggagggaaagcgccttttcctgcagctcttcagagccacagacctcagggtggagtgagcccatggtgggcagtgggcaaggcggtgggtggtgggcaaggtgggacctcctgcagcctggaaagaggagggaggccaaggccattccctaactccctcctgcccctggtctgaggaggagggactctggagtagcagaggggctgggaaagagggggcaggggctgctgggacactgagcaggagggaggcctgagcacactgctttggaaattattctaaacacaaaaaagggaaagaaaatgttatttctccctaagtcaggagcatgcagagctagcccacctcatgtccagctgtccactttccatcctggagaaagaacagtgtgcctcaaactcctgccctccccaggcctctggggcccactggaaagggctctgaccccctggcccagccgggctctctagtggtgatccggctcattctcctgcaagttggaagcacaattttccccccaagtggaggaaaaggaaagggccccagcctactgaagaggtgtttattttttaactaacagcctcccaccccattaagactcaccaggagaggtctgagggccattcagaacccactcctgagtgggtgggtgggtgggactcagtccagagacctaacattcagaatatagcattggttgcctattttgagatggatttaatctcccacagtattcatgagaccatctgatggaatcagatccctgagccaccttgcaggacgttttccccaacctcttacaccctggatgtcactttggaaaccaagcccttggaagcaagtggggtggcatgggagagaagggaggaggtgggcacaggtggtgagcttatgtgtgggcactctactgcctcacagaagccagccaagtgccaaggtcagcttggctggtctgaggccaccttcttagccaaaaacctagggttcattttcaggactttgataatgaacaacaaaatggggacttctttgggcagatgctaggtcagttgttttcacctaatatcctcttttagctgcatgtatatttatttataattataaccctggtggactgcagccttcatctttattgggaatgagtttgttataaatcagaaatgggtccatgatgaccactgttttccaaacccagtctgttccctgctccctcgctggcaagccccaccacacaggagtgaggccaggggctaggagttctaagaacagaggctggggtgagggtggcacccaggcagctgcatctggtctgttttaatttaactgtatttaatttgctttcaaaattaaaagtcaaatacagtttttaacagtcctaaaaaaaaaaaa

SEQ ID NO: 6 - DNA, 3666 bp, Homo sapiens
ggagggagccgcacgcggcccagggcagcggtctaggggcgccggggccggggcgtaggggccgttgcccgcgatggaccgcaccggagacgctccggactcgtcgccgcaggtgtccaccccccagggttcctgacccctgcccctggacagcgaccccttctcagactccagttgggccggactctccaaacctgcttccgcaatgggtgggttgtgagtgctggtaagacctgctagccaacattcagctgctctgtcctctccatgcctggccggcccggcccatgcctgttcttttctcccctgtgctgccgccgcccgtggccgcccctctcctgaacttaccgccactcaggtaatgaggagccgtgggtgcagccagccttggagatgccgaagagacgggacatcctagcgatcgtcctcatcgtgctgccctggactctgctcatcactgtctggcaccagagcaccctcgcacccctgctcgcggtacataaggatgagggcagtgacccccgacgcgaaacgccgcccggcgccgaccccagggagtactgcacgtctgaccgcgacatcgtggaggtggtgcgcaccgagtacgtgtacacgcggcccccgccatggtccgacacgctgcccaccatccacgtggtgacgcccacctacagccgcccggtgcagaaggccgagctgacgcgcatggccaacacgctgctgcacgtgcccaacctccactggctggtggtggaggatgcgccgcgccggacgccgctgaccgcgcgcctgctgcgcgacaccggcctcaactacacgcacctgcacgtggagacgccccgcaactacaagctgcgcggagacgcccgcgacccacgcatcccgcggggcaccatgcagcgcaacctggccctgcgctggctgcgcgagaccttcccgcgcaactccagccagcctggcgtggtctacttcgccgacgacgacaacacctacagcctggagctcttcgaagagatgcgcagcaccaggagggtgtccgtgtggcccgtcgccttcgtgggtggcctgcggtacgaggccccacgggtgaacggggcagggaaggtggtcggctggaagacggtgtttgacccccaccggccatttgcaatagacatggctggatttgccgtcaacctgcggctcattctgcagcgaagccaggcctacttcaagctgcgaggtgtgaagggaggctaccaggaaagcagcctccttcgagaacttgtcaccctcaacgacctggagcccaaggcagccaactgcaccaagatcctggtgtggcacacacggacagagaagccagtgctggtgaatgagggcaagaagggcttcactgacccctcggtggagatctgagcctcaggatgcaggagcctcctcctcagaccctgttcttggccttccatcctctccccacggctgatggtccctccaaggccgactcctaaggaatcaccatcaccctcctttctattctgggggcttctgagagagcccagcctgatgccagaacaaaggacagagaatttaagcacagaaatcccagacctgttgttctctccatccagcgtgaccagggcccgagagacctgatggccagggtggggtgtccagcaccagccaagctggtgctccagcgcacctccccagagctccccgcactgacggggctgcaggagcaggtgcagtgggcgcccacactggccctgcagtgatgcagggcgggagggagataagaagaccccgcagtcaagtggagcatggccctccctggctccctgtccctgggctcagcacgaccacacaggacacccagccagggaattctgaagaccagagagcagcccacgggcatcacgagcgctctgctcctctcctgggcccctgctcttcccgagagctgcccccaaatcagacatacctctgtggctctcctctggttcacgtttacagagcataaggctgtcttggatcccaacaggcacccagccctgcatggggggagcctgggcctaataggcaccccctgtacctcaggctgtggcgggagcagagtccccccctccggcccctcttcctttaccccttctcctccagcagtggcaaaggggtaggctctagagccagcacaggtcactgcctgacctggactaagaaccccacggccccactgtccacacactgcctccccaccgcccacctcggctgctaggcccctcgcctggactggactggggagggaaagcgccttttcctgcagctcttcagagccacagacctcagggtggagtgagcccatggtgggcagtgggcaaggcggtgggtggtgggcaaggtgggacctcctgcagcctggaaagaggagggaggccaaggccattccctaactccctcctgcccctggtctgaggaggagggactctggagtagcagaggggctgggaaagagggggcaggggctgctgggacactgagcaggagggaggcctgagcacactgctttggaaattattctaaacacaaaaaagggaaagaaaatgttatttctccctaagtcaggagcatgcagagctagcccacctcatgtccagctgtccactttccatcctggagaaagaacagtgtgcctcaaactcctgccctccccaggcctctggggcccactggaaagggctctgaccccctggcccagccgggctctctagtggtgatccggctcattctcctgcaagttggaagcacaattttccccccaagtggaggaaaaggaaagggccccagcctactgaagaggtgtttattttttaactaacagcctcccaccccattaagactcaccaggagaggtctgagggccattcagaacccactcctgagtgggtgggtgggtgggactcagtccagagacctaacattcagaatatagcattggttgcctattttgagatggatttaatctcccacagtattcatgagaccatctgatggaatcagatccctgagccaccttgcaggacgttttccccaacctcttacaccctggatgtcactttggaaaccaagcccttggaagcaagtggggtggcatgggagagaagggaggaggtgggcacaggtggtgagcttatgtgtgggcactctactgcctcacagaagccagccaagtgccaaggtcagcttggctggtctgaggccaccttcttagccaaaaacctagggttcattttcaggactttgataatgaacaacaaaatggggacttctttgggcagatgctaggtcagttgttttcacctaatatcctcttttagctgcatgtatatttatttataattataaccctggtggactgcagccttcatctttattgggaatgagtttgttataaatcagaaatgggtccatgatgaccactgttttccaaacccagtctgttccctgctccctcgctggcaagccccaccacacaggagtgaggccaggggctaggagttctaagaacagaggctggggtgagggtggcacccaggcagctgcatctggtctgttttaatttaactgtatttaatttgctttcaaaattaaaagtcaaatacagtttttaacagtcctaaaaaaaaaaaa

SEQ ID NO: 7 - protein, 334 aa, Homo sapiens
MPKRRDILAIVLIVLPWTLLITVWHQSTLAPLLAVHKDEGSDPRRETPPGADPREYCTSDRDIVEVVRTEYVYTRPPPWSDTLPTIHVVTPTYSRPVQKAELTRMANTLLHVPNLHWLVVEDAPRRTPLTARLLRDTGLNYTHLHVETPRNYKLRGDARDPRIPRGTMQRNLALRWLRETFPRNSSQPGVVYFADDDNTYSLELFEEMRSTRRVSVWPVAFVGGLRYEAPRVNGAGKVVGWKTVFDPHRPFAIDMAGFAVNLRLILQRSQAYFKLRGVKGGYQESSLLRELVTLNDLEPKAANCTKILVWHTRTEKPVLVNEGKKGFTDPSVEI

SEQ ID NO: 8 - RNA, 21 nt, Artificial Sequence
  Artificial Primer
gcugauuguucuaaauggauu

SEQ ID NO: 9 - RNA, 21 nt, Artificial Sequence
  Artificial Primer
uccauuuagaacaaucagcuu

SEQ ID NO: 10 - RNA, 21 nt, Artificial Sequence
  Artificial Primer
guaagagaucccuucgaaauu

SEQ ID NO: 11 - RNA, 21 nt, Artificial Sequence
  Artificial Primer
uuucgaagggaucucuuacuu

SEQ ID NO: 12 - RNA, 21 nt, Artificial Sequence
  Artificial Primer
ugacaaccaugccggagguuu

SEQ ID NO: 13 - RNA, 21 nt, Artificial Sequence
  Artificial Primer
acuuccggcaugguugucauu

SEQ ID NO: 14 - RNA, 21 nt, Artificial Sequence
  Artificial Primer
cuagcaaguucaucacguuuu

SEQ ID NO: 15 - RNA, 21 nt, Artificial Sequence
  Artificial Primer
aacgugaugaacuugcuaguu

(19) United States
(12) Patent Application Publication
(10) Pub. No.: US 2014/0100262 A1
(43) Pub. Date: Apr. 10, 2014

(54) Methods and Compositions for Maintaining Blood-Brain Barrier Integrity

(71) Applicant: Georgia Regents Research Institute, Inc., Augusta, GA (US)

(72) Inventors: Robert K. Yu, North Augusta, SC (US); Somsankar Dasgupta, North Augusta, NC (US)

(73) Assignee: Georgia Regents Research Institute, Inc., Augusta, GA (US)

(21) Appl. No.: 14/047,585

(22) Filed: Oct. 7, 2013

Related U.S. Application Data
(60) Provisional application No. 61/710,693, filed on Oct. 6, 2012.

(51) Int. Cl. A61K 31/713

(52) U.S. Cl. CPC A61K 31/713
USPC 514/44 A; 435/375

(57) ABSTRACT

Methods of maintaining or improving blood-brain barrier integrity and increasing resistance to cytokine-induced cell permeability are disclosed. It has been discovered that down-regulating the expression or production of sulfoglucuronyl glycolipids, for example SGPG, in endothelial cells of the blood-brain barrier or the blood-nerve barrier reduces apoptosis of these endothelial cells and thereby promotes the integrity of the barriers. Promoting the integrity of these barriers includes, but is not limited to reducing or inhibiting passage of immune cells, pathogenic immunoglobins, or bio-degrading molecules across the blood-brain barrier or blood-nerve barrier into the nervous system. Down-regulating expression or production or SGPG also increases the resistance of the endothelial cells to cytokine-induced cell permeability.

### CROSS-REFERENCE TO RELATED APPLICATION

This application claims benefit of and priority to U.S. Provisional Application No. 61/710,693 filed Oct. 6, 2012, which is hereby incorporated herein by reference in its entirety.

### STATEMENT REGARDING FEDERALLY SPONSORED RESEARCH OR DEVELOPMENT

This invention was made with government support under grant numbers NS11853 and NS26994, awarded by the National Institutes of Health. The government has certain rights in the invention.

### REFERENCE TO SEQUENCE LISTING

The Sequence Listing submitted Oct. 6, 2013 as a text file named “GRU_{—}2011_{—}028_ST25.txt,” created on Oct. 6, 2013, and having a size of 26,722 bytes is hereby incorporated by reference pursuant to 37 C.F.R. §1.52(e)(5).

### FIELD OF THE INVENTION

The field of the invention is generally related to compositions and methods for maintaining the integrity of the blood-brain barrier and blood-nerve barrier during inflammation.

### BACKGROUND OF THE INVENTION

Glycosphingolipids (GSLs) are important constituents of the plasma membrane and are involved in regulating a variety of cellular functions (Dasgupta S, et al., J. Neurosci. Res., 85:1086-1094 (2007); Hakomori S I, Biochim. Biophys. Acta., 1780:325-346 (2008); Kanda T, et al., Proc. Natl. Acad. Sci. USA, 92:7897-7901 (1995)). A large number of glycoproteins, such as neural cell adhesion molecules (NCAMs) (Ong E, et al., J. Biol. Chem., 277:18182-18190 (2002)), L1, myelin-associated glycoprotein (MAG) (Kruse J, et al., Nature, 316:146-148 (1985)), tenascin-C, tenascin-R, and tissue plasminogen activator (Voshol H, et al., J. Biol. Chem., 271:22957-22960 (1996)), contain the human natural killer antigen (HNK-1) epitope, a carbohydrate antigen that modulates neurite outgrowth (Martini R, et al., Eur. J. Neurosci., 4:628-639 (1992)), cell adhesion, and synaptic plasticity (Dityatev A, et al, Nat. Rev. Neurosci., 4:456-468 (2003)). The minimal structural components of the HNK-1 epitope have been shown to consist of a sulfated disaccharide residue, 3-sulfoglucuronosyl (β1-3) galactosyl (β1-) (Tokuda A, et al., J. Carbohyd. Chem., 17:535-546 (1998)). The HNK-1 epitope is also shared by two glucuronosyl glycosphingolipids (SGGLs), sulfated glucuronosyl paragloboside (SGPG) and sulfated glucuronosyl lactosaminyl paragloboside (SGLPG).

Guillian-Barre Syndrome (GBS) is likely triggered by infection by Gram-negative bacteria, such as Campylobacter jejuni, through a molecular mimicry mechanism in which anti-glycolipid antibodies are generated against an oligosaccharide portion of the bacterial lipooligosaccharide coat (Yu R K, et al., Infect. Immun., 2006; 74:6517-6527 (2006)). Clinical symptoms develop by two principal pathogenic mechanisms: a) the autoantibodies, such as antibodies against SGPG, must enter from the circulation into the nerve parenchyma to cause neurodegeneration by an antibody-mediated and complement dependent mechanism (Maeda Y, et al., Brain Res., 541:257-264 (1991a); Maeda Y, et al., Exp. Neurol., 113:221-225 (1991b); Kohriyama T, et al., J. Neurochem., 51:869-877 (1988); Kaida K, et al., Glycobiology, 19:676-692 (2009); Kohriyama T, et al., J. Neurochem., 48:1516-1522 (1987)), and b) by a cell-mediated process that entails the penetration of inflammatory T cells, elicited by bacterial infection, to enter into the nerve tissues (Ariga T, et al., J. Lipid Res., 28:285-291 (1987); Dasgupta S, et al., J. Neurosci. Res., 85:1086-1094 (2007); Kanda T, et al., Proc. Natl. Acad. Sci. USA, 92:7897-7901 (1995); Ariga T, et al., J. Lipid Res., 28:285-291 (1987); Kohriyama T, et al., J. Neurochem., 51:869-877 (1988)). In either case, the blood-brain and blood-nerve barrier (BBB/BNB) function is compromised to allow immunoglobulins or immune cells to penetrate the nerve parenchyma to attack the nerve tissues (Yu R K, et al., Infect. Immun., 2006; 74:6517-6527 (2006); Kohriyama T, et al., J Neurochem., 48:1516-1522 (1987)). At present, although the precise etiology of disease onset is still not fully understood (Geleijns K, et al., Neurology, 64:44-49 (2005); Compston A, et al., Lancet., 372:1502-1517 (2008)), the detection of a large concentration of inflammatory cytokines, presence of lymphocytes in nervous tissues, and an elevated concentrations of autoantibodies in the patient serum and body fluid is a hallmark of GBS.

Two inflammatory cytokines, TNFα and IL-1β, presumably elicited by bacterial infection, up-regulate SGPG expression in bovine brain endothelial cells (BMECs) and in human cerebromicrovascular endothelial cells (SV-HCECs). These cytokines promote CD4+ cell adhesion to endothelial cells with SGPG serving as a ligand for L-selectin (Dasgupta S, et al., J. Neurosci. Res., 87:3591-3599 (2009); Dasgupta S, et al., J. Neurosci. Res., 85:1086-1094 (2007)) expressed on T cells. Subsequent studies revealed that both TNFα and IL-1β stimulated glucuronosyltransferase genes, both the P and S forms, designated as GLcATp and GLcATs, respectively, and as such up-regulation was mediated via stimulation of NF-κB activity. Inhibition of HNK-1ST gene expression, using HNK-1 sulfotransferase siRNA or HNK-1STsiRNA, down-regulates NF-κB activity and, consequently, blocks cytokine-mediated SGPG elevation and T cell adhesion (Dasgupta S, et al., J. Neurosci. Res., 87:3591-3599 (2009)) and penetration through the tight junction. The adhesion ultimately leads to the penetration of lymphocytes and other active agents into the brain and nerve compartments, which triggers phagocytosis, demyelination, and axonal degeneration. The regulation of cytokine-mediated changes in endothelial cells and the mechanism of penetration of a large number of T cells through the tight-gap junction is a subject of considerable scientific interest.

Although these studies indicate that SGPG is a direct participant in inflammatory processes as an adherent for T cells, the mechanism of action of SGPG up- and down-regulation in endothelial cell function, and as an active signaling mediator in endothelial cell death has not yet been examined.

### SUMMARY OF THE INVENTION

Methods of maintaining or improving blood-brain barrier integrity and increasing resistance to cytokine-induced cell permeability are disclosed. It has been discovered that down-regulating the expression or production of sulfoglucuronyl glycolipids, for example SGPG, in endothelial cells of the blood-brain barrier or the blood-nerve barrier reduces apoptosis of these endothelial cells and thereby promotes the integrity of the barriers. Promoting the integrity of these barriers includes, but is not limited to reducing or inhibiting passage of immune cells, pathogenic immunoglobins, or bio-degrading molecules across the blood-brain barrier or blood-nerve barrier into the nervous system. Down-regulating expression or production or SGPG also increases the resistance of the endothelial cells to cytokine-induced cell permeability.

Certain embodiments provide compositions and methods for promoting the integrity of the blood-brain barrier or the blood-nerve barrier by administering to a subject in need thereof an effective amount of antagonist of one or more of the enzymes in the metabolic pathway for producing SGPG. Exemplary enzymes in the pathway for producing SGPG that can be antagonized include but are not limited to glucuronosyltransferases and killer epitope-1 sulfotransferase (HNK-1ST). Representative glucuronosyltransferases include, but are not limited to GlcATp and GlcATs. Antagonists include but are not limited to small molecule antagonists that inhibit enzymatic activity or inhibitory nucleic acids including, but not limited to siRNA, anti-sense nucleic acids, or combinations thereof.

### BRIEF DESCRIPTION OF THE DRAWINGS

FIGS. 1A, B, and C show the activation of ERK and inhibition of IKB and caspase 3 by HNK-1 siRNA transfection. FIG. 1A is a western blot showing the inhibitory effects of different siRNAs on caspase 3, pIKB, tIKB and tubulin. Inhibition of caspase 3 and IKB is shown. FIG. 1B is a western blot showing the activation effects of different siRNAs on pERK, tERK and tubulin. Activation of ERK is shown. Cells were exposed to cytokines overnight (between 18-24 h) and protein was dissolved using Lemmelli buffer. The protein bands were separated and identified using specified antibody. Scr, Scrambled siRNA transfection; HNK-1si, HNK-1ST siRNA transfection; Scr+T, Scrambled siRNA+TNFα; HNK-1 si+T, HNK-1ST siRNA+TNFα; Scr+1, Scrambled siRNA+IL-1β; HNK-1 si+I, HNK-1ST siRNA+IL-1β. FIG. 1C is a bar graph showing the quantification of bands on the western blot based on the ratio of pERk/tERK, pIKB/tIKB, and caspase 3/tubulin. Each band (area) was scanned and quantified using the ImageJ program. The ratio of two band area such as pERk/tERK, pIKB/tIKB, and caspase 3/tubulin was determined and plotted. Bar represents SD from at least 3 independent experiments, Comparison was made by T-test analysis between each set, Scr vs. HNK-1si, p<0.005; Scr+T vs. HNK-1si+T, p<0.0005; Scr+I vs. HNK-1si+I, p<0.0005. Comparison between groups using two-way ANOVA indicates the significance with a p value of 1.59×10-10.

FIG. 2 is a western blot of SV-HCECs incubated with TNFα and probed with antibodies to pERK, tERK and Tubulin. It shows a time-course study of ERK activation in SV-HCECs by a cytokine Cells were incubated with TNFα (100 ng/ml) at different time periods from 0 h (control) to 24 h. Cells were collected and proteins were dissolved in Lemmili's buffer.

FIG. 3 shows an immunohistochemistry analysis of cells treated with IL-1β and TNF-α after being exposed to different siRNAs. The immunohistochemistry shows the protection from IL-1β- and TNFα-iated cell death (TUNEL) by HNK-1ST siRNA transfection. Cells were exposed to IL-1β (A-D) and TNFα (E-H) after scrambled and HNK-1ST siRNA transfection. A, C, E, and G present Hoechst staining; B, D, F, and H are TUNEL assays. A, B, E, and F are HNK-1ST siRNA transfection; C, D, G, and H represent scrambled siRNA transfection. Cell death was observed in scrambled transfection after cytokine exposure (D and H).

FIG. 4 shows a TLC-immunooverlay of SGPG isolated from SV-HCECs after EGFP, EGFP-GlcATp, EGFP-GlcATs and EGFP-GlcATp+Ts transfection. The transfected cells were incubated for 48 h. Lipids were extracted using solvent mixtures and SGPG was purified into a fraction using DEAE-Sephadex A25 column. The fraction was dissolved in a defined volume of solvent (chloroform:methanol:water 12:7:1, v/v) according to protein concentration, and a portion of the solution equivalent to an equal amount of protein was applied on an aluminum-backed HPLTC along with a reference standards. The plate was developed in chloroform:methanol:0.25% CaCl2 (50:45:10 v/v). After the coating with an isobutylmethacrate solution in hexane, the plate was incubated with the mAb NGR50 followed by a secondary HRP-conjugated anti-mouse IgG. The bands were identified using ECL. Band from each lane was scanned and quantitated using the ImageJ program.

FIG. 5 is a bar graph of relative NFkB activity (%) versus vector, GlcATp, GlcATs and GlcATp+s transfected endothelial cells. Vector, green fluorescent protein; GlcATp, GlcATp transfection; GlcATs, GlcATs transfection, GlcATp+s, combined transfection of GlcATp and GlcATs. Bar represents SD from four independent determinations. * p<0.05; ** p<0.005. Comparison was made between vector vs. GlcATp, p<0.05; vector vs. GlcATs, p<0.005; vector vs. GlcATp+s, p<0.005.

FIGS. 6A, 6B, and 6C show the activation and inhibition of IKB phosphorylation and ERK and Akt phosphorylation by SGPG in SV-HCECs. FIG. 6A Caspase 3 activation and inhibition of IKB phosphorylated by SGPG in SV-HCECs; (B) Inhibition of ERK and Akt phosphorylation by SGPG in SV-HCECs. GFP, Green fluorescent protein; GlcATp, GlcATp transfection; GlcATs, GlcATs transfection, GlcATp+s, Combined transfection of GlcATp and GlcATs. FIG. 6C is a bar graph showing the quantification of bands on the western blot based on the ratio of two band areas such as caspase 3/tubulin, pIKB/tIKB, pERk/tERK, and pAKt/tAkt in cells transfected with different agents. Each band (area) was scanned and quantified using ImageJ program. The error bars represent SD from four independent assays. *** p<0.0005. Comparison was made between GFP vs. GlcATp; GFP vs. GlcATs; and, GFP vs. GlcATp+Ts; in all cases p<0.0005. Note: The expression of caspase 3/tubulin in GFP transfected cells was negligible.

FIGS. 7A and 7B show western blots of TNFR1, TNFR2 and tubulin expression. FIG. 7A SGPG is a western blot analysis showing that SGPG stimulates TNFR1 expression but inhibits TNFR2 expression. FIG. 7B is a western blot analysis showing the effect on TNFR1 and TNFR2 expression by silencing HNK-1ST gene. GFP, Green fluorescent protein; GlcATp, GlcATp transfection; GlcATs, GlcATs transfection, GlcAT p+s, Combined transfection of GlcATp and GlcATs; Scr, Scrambled siRNA transfection; HNK-1si, HNK-1ST siRNA transfection.

FIG. 8 shows the immunocytochemical localization of GFP, SGPG, and caspase 3 expression in GFP, GlcATp, GlcATs and GlcATp+s-transfected SV-HCECs. Cells were stained with MAb NGR50 (SGPG, cy3) and caspase 3 antibody (cy 5) and visualized under a confocal microscope. Panel 1: GFP expression, Panel 2: SGPG expression, Panel 3: Caspase 3 expression, Panel 4 represents the merging of panel 2 and panel 3. GFP, Green fluorescent protein; GlcATp, GlcATp transfection; GlcATs, GlcATs transfection, GlcATp+s, combined transfection of GlcATp and GlcATs.

FIG. 9 shows a bar graph of viable cells (in percent) versus cells transfected with GFP, GlcATp, GlcATs, and GlcATp+s. Determination of cell viability by FLICA in SV-HCECs Cells were grown on coated cover-slips in a 12-well plate, stained with FLICA and Hoechst, respectively, fixed and visualized under a fluorescent microscope. FLICA-positive cells (red) were counted compared to total cells (Hoechst). GFP, Green fluorescent protein; GlcATp, GlcATp transfection; GlcATs, GlcATs transfection, GlcATp+s, Combined transfection with GlcATp and GlcATs. Bar means the SD from a set of three independent assays. *** p<0.0005 Comparison was made between GFP vs. GlcATp, p<0.05; GFP vs. GlcATs, p<0.005; and, GFP vs. GlcATp+s, p<0.0005.

FIG. 10 is a diagrammatic presentation of the SGPG-mediated novel signaling pathway for endothelial cell death and survival. Inflammatory cytokines up-regulate SGPG expression by stimulating GlcATp, GlcATs, and HNK-1ST genes via NF-κB activation. SGPG over-expression stimulates lymphocyte (T cell) adhesion (Dasgupta S, et al., J Neurosci Res., 87:3591-3599 (2009)) and promotes endothelial cell death, and thereby increases the cell permeability. Silencing of SGPG expression (by HNK-1ST siRNA) inhibits NF-κB activity, stimulates ERK activation, and prevents endothelial cell death.

### DETAILED DESCRIPTION OF THE INVENTION

### I. Definitions

As used herein, “treat” means to prevent, reduce, decrease, or ameliorate one or more symptoms, or characteristics of cancer, in particular ovarian cancer, to halt the progression of one or more symptoms, or characteristics of ovarian cancer.

The terms “individual,” “subject,” and “patient” are used interchangeably herein, and refer to a mammal, including, but not limited to, rodents, simians, and humans.

The terms “reduce”, “inhibit”, “alleviate” and “decrease” are used relative to a control. One of skill in the art would readily identify the appropriate control to use for each experiment. For example a decreased response in a subject or cell treated with a compound is compared to a response in subject or cell that is not treated with the compound.

As used herein, the terms “inhibitors” or “antagonists” refers to compounds or compositions that directly or indirectly partially or totally block activity, decrease, prevent, delay activation, inactivate, desensitize, or down regulate the activity or expression of the targeted molecule. Antagonists are, for example, polypeptides, such as antibodies, as well as nucleic acids such as siRNA or antisense RNA, as well as naturally occurring and synthetic antagonists, including small chemical molecules.

An “immune cell” refers to any cell from the hemopoietic origin including but not limited to T cells, B cells, monocytes, dendritic cells, and macrophages.

As used herein, the term “polypeptide” refers to a chain of amino acids of any length, regardless of modification (e.g., phosphorylation or glycosylation). The term polypeptide includes proteins and fragments thereof The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

The term “endogenous” with regard to a nucleic acid or protein refers to nucleic acids or proteins normally present in the host.

The term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompass “exogenous” and “non-native” elements.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

### II. Methods of Maintaining Blood-Brain Barrier and Blood-Nerve Integrity

Sulfoglucuronyl glycolipids (SGGLs) are found on the surface of endothelial cells and can be upregulated by inflammatory cytokines such as TNF-α and IL-1β. The upregulation of SGGLs in endothelial cells can lead to increased T cell adhesion because the SGGLs serve as a ligand for L-selectin found on T cells and are used in T cell migration. The upregulation of SGGLs in endothelial cells at the blood brain barrier can increase T cell adhesion and migration into the brain. Downregulation of SGGLs reduces T cell adhesion and reduces T cell migration into the brain. Thus, antagonists of TNF-α and IL-1β can be used to reduce T cell adhesion and T cell migration into the brain by interfering with the biological activity of TNF-α and IL-1β on endothelial cells of the blood-brain barrier or blood-nerve barrier.

It has also been discovered that upregulation of SGGLs can lead to increased apoptosis of the endothelial cells. Apoptotic endothelial cells can result in a weakened blood-brain barrier (BBB) or nerve-brain barrier (NBB) therefore allowing unwanted cells, such as inflammatory cells, into the brain causing neuroinflammation. It has also been discovered that reducing the expression or production of SGGLs can maintain or improve the integrity of the BBB, the NBB, or combinations thereof. Accordingly, methods and compositions for maintaining or improving the integrity of the BBB, the NBB, or combinations by inhibiting or reducing the expression or production of SGGLs and thereby reducing apoptosis of endothelial cells and reducing the ability of immune cells to enter the brain are disclosed.

Two types of SGGLs are known, sulfated glucuronosyl paragloboside (SGPG) and sulfated glucuronosyl lactosaminyl paragloboside (SGLPG), whose structures were established independently by two groups (Chou D K, et al., J. Biol. Chem., 261:11717-11725 (1986); Ariga T, et al., J. Lipid Res., 28:285-291 (1987)). The structures of these two SGGLs are represented as follows: SGPG, SO4-3GlcA(β1-3)Gal(β1-4)GlcNAc(β1-3)Gal(β1-4)Glc(β1-1) ceramide; and SGLPG, SO4-3GlcA(β1-3)Gal(β1-4)GlcNAc(β1-3) Gal(β1-4)GlcNAc(β1-3)Gal(β1-4)Glc(β1-1) ceramide. Both SGGLs are minor components of the total GSLs of central and peripheral nervous systems (CNS and PNS), with SGPG being the major component of the two (Ariga T, et al., J. Biol. Chem., 262:848-853 (1987); Ariga T, et al., J. Lipid Res., 28:285-291 (1987), Chou D K, et al., J. Biol. Chem., 261:11717-11725 (1986)). In addition to their known biological functions in nervous system development, they are also involved as autoantigens in autoimmune peripheral neuropathies such as Guillian-Barré syndrome (GBS).

Therefore, in some embodiments, methods and compositions for maintaining or improving the integrity of the BBB, the NBB, or combinations by inhibiting or reducing the expression or production of SGPG, SGLPG, or combinations thereof. Expression or production of SGPG or SGLPG can be inhibited or reduced by inhibiting or reducing expression of compontent of SGPG or SGLPG biosynthetic pathways. In a preferred embodiment, expression of SGPG is reduced by inhibiting or reducing expression of a glycosyltransferases involved in the biosynthesis of SGPG.

A. Inhibition of HNK-1ST Gene

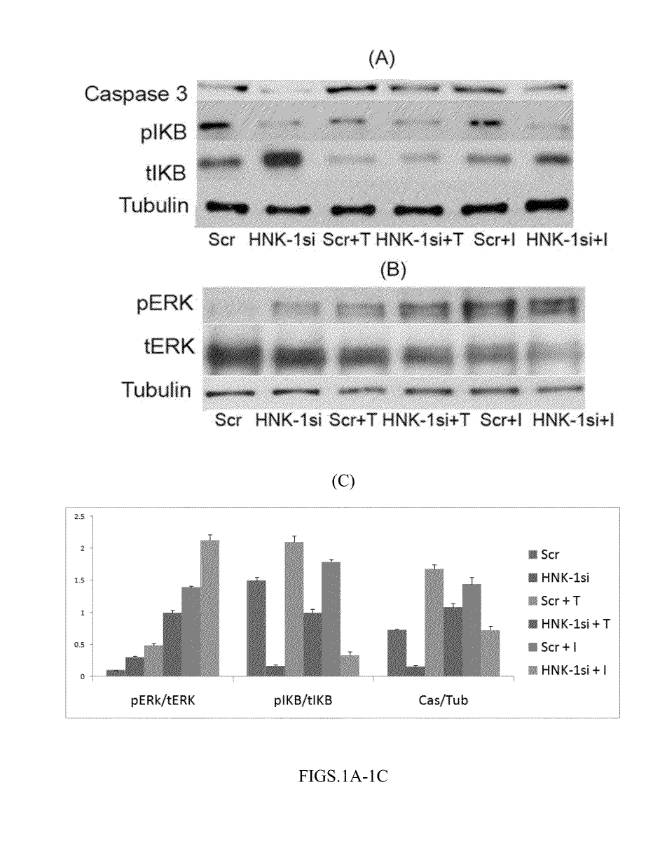

In some embodiments, the methods of inhibiting or reducing the expression or production of SGPG including inhibiting or reducing expression of human natural killer-1 sulfotransferase (HNK-1ST). HNK-1ST, also designated carbohydrate sulfotransferase 10 (CHST10), is a Golgi-associated sulfotransferase that functions in the biosynthesis of HNK-1, a neuronally expressed carbohydrate that harbors a sulfoglucuronyl residue. HNK1-ST catalyzes the transfer of sulfate to position 3 of terminal glucuronic acid of both protein- and lipid-linked oligosaccharides. HNK-1ST and glucuronosyltransferase P (GLCATP) expression is necessary to form the HNK-1 carbohydrate epitope on the cell adhesion molecule NCAM. The deduced 356 amino acid type II transmembrane protein contains three potential N-glycosylation sites and a conserved RDP sequence that is also present in other Golgi-resident sulfotransferases. The data provided in the Examples shows that cytokine-mediated SGPG up-regulation preceded via NF-κB activation, as evidenced by phosphorylation of IKB, leading to apoptosis by stimulating caspase 3 activity and inhibiting ERK activation. Inhibition of HNK-1ST gene expression down-regulated the NF-κB activity by inhibiting IKB phosphorylation, and reducing caspase 3 activity. At the same time, the ERK survival pathway, but not Akt, was also activated (FIG. 1). Therefore, the combined effect of inhibiting or reducing HNK-1ST can down regulate SGPG and offer the cell protection from undergoing apoptosis and resistance in cytokine-stimulated cell permeability changes.

A coding sequence for HNK-1ST can be:

| | |
|---|---|
| 1 | aggcggcgcg cacggccgga taggcgcgag ggggccgcgt gaggcggtgc cggcgttctg |
| 61 | gcccccaaag ccggtctagc gcgccgggcg tcttccttac ttccgctgcc gccgccgcca |
| 121 | catcccggga cccgacgggc cgcggcgcgg aggcctcggg gcaaggtggg gcgggcctcc |
| 181 | cgagctccca ggaccccgcg cgcttcgccc acaggcccgg cgaagcccga cccgcgcggc |
| 241 | gcccccaggg ccaggggagg agcctaagga cccggacgag cgccgctcca gtaggtgaca |
| 301 | agaggaacca agaacctcag ttcaggggaa acacagcaag gaaatgtgag ccccaggctg |
| 361 | cagaaggaag agtcagtgaa tggctgcggt gtgacaacat gcaccaccag tggcttctgc |
| 421 | tggccgcatg cttttgggtg attttcatgt tcatggtggc tagcaagttc atcacgttga |
| 481 | cctttaaaga cccagatgtg tacagtgcca aacaggagtt tctgttcctg acaaccatgc |
| 541 | cggaagtgag gaagttgcca gaagagaagc acattcctga ggaactgaag ccaactggga |
| 601 | aggagcttcc agacagccag ctcgttcagc ccctggtcta catggagcgc ctggaactca |
| 661 | tcagaaacgt ctgcagggat gatgccctga agaatctctc gcacactcct gtctccaagt |
| 721 | ttgtcctgga ccgaatattt gtctgtgaca agcacaagat tcttttctgc cagactccca |
| 781 | aagtgggcaa cacccagtgg aagaaagtgc tgattgttct aaatggagca ttttcttcca |
| 841 | ttgaggagat ccccgaaaac gtggtgcacg accacgagaa gaacggcctt cctcggctct |
| 901 | cttccttcag tgatgcagaa attcagaagc gattgaaaac atacttcaag ttttttattg |
| 961 | taagagatcc cttcgaaaga cttatttctg catttaagga taaatttgtt cacaatcccc |
| 1021 | ggtttgagcc ttggtacagg catgagattg ctcctggcat catcagaaaa tacaggagga |
| 1081 | accggacaga gacccggggg atccagtttg aagatttcgt gcgctacctc ggcgatccga |
| 1141 | accacagatg gctagacctt cagtttgggg accacatcat tcactgggtg acgtatgtag |
| 1201 | agctctgtgc tccctgtgag ataatgtaca gtgtgattgg acaccacgag accctggagg |
| 1261 | acgatgcccc atacatctta aaagaggctg gcattgacca cctggtgtca tacccgacta |
| 1321 | tccctccggg cattaccgtg tataacagaa ccaaggtgga gcactatttc ctgggcatca |
| 1381 | gcaaacgaga catccgacgc ctgtatgccc gtttcgaagg ggactttaag ctctttgggt |
| 1441 | accagaaacc agactttttg ctaaactaat gcataagacc tatgaattca aatatcttta |
| 1501 | ttagacctgg ggctaaccag gtgaagatct gagcccagaa atgacccttc ctccaccaca |
| 1561 | cccctccttt gaggacgccc ggggtctccc acaggcctgt gagttgcctc ggcatatgac |
| 1621 | gcagaacccc aactgttaca acttagtttg gatgtaagat gctctgagga ccctgcccac |
| 1681 | acccctgcgt gcattaggat gtcgctggcc tttgctcacc tcagagggga gaaaaggcta |
| 1741 | aagatttgca gtttgacagc ccagcaggga ggaagcatca cacagcgtta ggagccgttt |
| 1801 | ccttcaggtg ttaaggaagg ggatgcccct gaggttctcc tggctagtca gggtggcttc |
| 1861 | acccatcact ggtgggttgc aggaacagca cccaggactc tgaggaggga cagagaagca |
| 1921 | agggggctgc tgaaatcgca gagacttttg cagcatcaga tctgaggagt aaaacggcac |
| 1981 | ctctggcctt catcttggtg ctgcgacaat tgtggaggca aagcattctt tctgtgacta |
| 2041 | ttttgttcct gtagacagtc agcgatggcc agagggtggt gtggtgtcca ggggtccatc |
| 2101 | tttccagaat ccatgcctgt gtaatgctgg tccatgcttc tgaacctgtg tctgccaagc |
| 2161 | gcctatttca ttcagcacaa gacatacgat tttagaaggt gaggggaggg gaggcttttt |
| 2221 | ctacctgaga aggggagtgt ctttgagggc cttaaaagga ccatggccca ggaatggggg |
| 2281 | cgctggttgg gcttggagct caggctgctg tggatcccgg cgcatcagtt ctgacttgcc |
| 2341 | ttacctgggt ggacagcagt gaatctccac ctgtcttctc cagggagctc ccatgttggg |
| 2401 | gctgaagacg agcaggggca acctgccagc atcacagaat tcagtgtagt ttatacattt |
| 2461 | cgattccttt catctcagca aaatgggcac tgccagagcc atttctgatc acaccaccat |
| 2521 | cctggaccat gtgactggaa ggtgggtaac caagttcacc agcaataaaa cccagcgccc |
| 2581 | aggtagcctc cagcagtgcg gcttcctggc aacaaggtag gccctggtgc agggcaagcc |
| 2641 | gcagcgacca tttcagatac cgtccacagc caggaccgct gagaactggg acagtttcct |
| 2701 | gggatgagtg ccagcctgag cctgcatggt gccgccgagc ccggggtgga ggagggagcc |
| 2761 | aggcttcgct tcaaggcggc ctctaccttt tctcagaatg gtttcctgat tgtgtcaatg |
| 2821 | tgaaagttaa ataaaattta tgtgccaaac ctgaaaaaaa aaaaaaaaaa aa |

(SEQ ID NO:1) (Homo sapiens carbohydrate sulfotransferase 10 (CHST10), mRNA, NCBI Reference Sequence: NM_{—}004854.4), which encodes a protein having the amino acid sequence:

MHHQWLLLAACFWVIFMFMVASKFITLTFKDPDVYSAKQEFLFLTTMPEVRKLPEEKHIPEELKPTGKELP
DSQLVQPLVYMERLELIRNVCRDDALKNLSHTPVSKFVLDRIFVCDKHKILFCQTPKVGNTQWKKVLIVLN
GAFSSIEEIPENVVHDHEKNGLPRLSSFSDAEIQKRLKTYFKFFIVRDPFERLISAFKDKFVHNPRFEPWY
RHEIAPGIIRKYRRNRTETRGIQFEDFVRYLGDPNHRWLDLQFGDHIIHWVTYVELCAPCEIMYSVIGHHE
TLEDDAPYILKEAGIDHLVSYPTIPPGITVYNRTKVEHYFLGISKRDIRRLYARFEGDFKLFGYQKPDFLL
N

### (SEQ ID NO:2).

B. Glucuronosyltransferase (GLcAT)

In some embodiments, the methods of inhibiting or reducing the expression or production of SGPG includes inhibiting or reducing expression of a glycosyltransferase. Glycosyltransferases are enzymes that are responsible for the biosynthesis of disaccharides, oligosaccharides and polysaccharides. They catalyse the transfer of monosaccharide moieties from activated nucleotide sugar (also known as the “glycosyl donor”) to a glycosyl acceptor molecule, usually an alcohol. There are several types of glycosyltransferases, one of which is the group of glucuronosyltransferases.

Glucuronosyltransferases are responsible for the process of glucuronidation (addition of glycosyl group from a UTP-sugar to a small hydrophobic molecule), a major part of phase II metabolism. Two forms of glucuronosyltransferase genes are the glucuronosyltransferase —S and —P genes (GLcATs and GLcATp, respectively).

The upregulation of SGGLs by inflammatory cytokines can be attributed to the cytokines initially upregulating expression of GLcAT's that result in an upregulation of SGGLs. Therefore, regulating GLcAT expression can have a downstream effect on SGGL expression and ultimately affect the endothelial cells expressing SGGLs as well as SGGLs dependent pathway such as endothelial cell apoptosis and immune cell infiltration across the BBB and BNB.

I. GLcATs

In some embodiments, the methods of inhibiting or reducing the expression or production of SGPG include inhibiting or reducing expression of a Glucuronosyl transferase-S. Glucuronosyltransferase-S (GLcATs), also known as B3GAT2 (beta-1,3-glucuronyltransferase 2) is expressed in many locations including, spinal cord, hippocampus and other brain regions, and, at lower levels in testis and ovary. GLcATs is involved in the biosynthesis of human natural killer antigen (HNK-1) carbohydrate epitope, which is implicated in cellular migration and adhesion in the nervous system. GLcATs catalyzes the transfer of a beta-1,3 linked glucuronic acid to a terminal galactose in different glycoproteins or glycolipids containing a Gal-beta-1-4GlcNAc or Gal-beta-1-3GlcNAc residue. Inflammatory cytokines, such as TNFα and IL-1β, stimulate GLcATs gene expression in the brain. An increase in GLcATs gene expression can promote T cell adhesion via SGPG-L-selectin recognition, which can be a preliminary step in neuroinflammatory disorders.

The coding sequence for GLcATs can be

| | |
|---|---|
| 1 | ctttctttcc ttgctttggg atcttgctgc tggatccgga gaggttctga gaagacaaga |
| 61 | gcaagggact gagagcaggc ttccgctgcg gcgcgcgaac acagccggga cacaaccccc |
| 121 | agcgtctcca cccgctcctc gccaccccgg cgggaatgtg aggaaggaaa gcccccagcg |
| 181 | ccgccgcccg ccctcgaagg cgtcccagag agcgtcctgg gggcccgcgg ctggagccct |
| 241 | tgtgcccgca gcaccgccgg actggagcgg cgaggcgcac cgggtgccgc ttctcggctt |
| 301 | ccactcttca gaaagagcgc ggtggggatc agcgcctttc ccgcactcgg cacaactccg |
| 361 | ggaccggcgg cgcgcggctg gaccgagtcc cgcttcccgc cagctcacct ggagtcgggg |
| 421 | gcagcccctg cccgcccgcc tgcacccctt gtcgctctag cttgcgcgaa cctgccgctc |
| 481 | ctccacgccc aggtagtgag ccccgcggct ccaggtctct gcagcgccct cggccccatg |
| 541 | gacagcgcac ccatcaccac tccctaagtg ctggcgccgc cgctgtccaa gctgcgcact |
| 601 | ggggtccctc ggctcgcccc tctctggggt gtccgagagg ccagggagcg tgcaccatga |
| 661 | agtccgcgct tttcacccgc ttctttatcc tcctgccctg gatcctaatt gtcatcatca |
| 721 | tgctcgacgt ggacacgcgc aggccagtgc ccccgctcac cccgcgcccc tacttctctc |
| 781 | cctacgcggt gggccgcggg ggcgcccgac tcccgctccg caggggcggc ccggctcacg |
| 841 | ggacccaaaa gcgcaaccag tctcggccgc agccacagcc ggagccgcag ctgcccacca |
| 901 | tctatgccat cacgcccacc tacagccgcc cggtgcagaa agcggagctg acccgcctgg |
| 961 | ccaacacgtt ccgccaggtg gcgcagctgc actggatcct ggtggaggac gcggcggcgc |
| 1021 | gcagcgagct ggtgagccgc ttcctggcgc gggccgggct gcccagcact cacctgcacg |
| 1081 | tgcccacgcc gcggcgctac aagcggcccg ggctgccgcg cgccactgag cagcgcaacg |
| 1141 | cgggcctcgc ctggctgcgc cagaggcacc agcaccagcg cgcgcagccc ggcgtgctct |
| 1201 | tcttcgctga cgacgacaac acctatagtc tggagctctt ccaggagatg cgaaccaccc |
| 1261 | gcaaggtctc cgtctggcct gtgggcctgg ttggtgggcg gcgctacgaa cgtccgctgg |
| 1321 | tggaaaacgg caaagttgtt ggctggtaca ccggctggag agcagacagg ccttttgcca |
| 1381 | tcgacatggc aggatttgct gtaagtcttc aagtcatttt gtccaatcca aaagctgtat |
| 1441 | ttaagcgtcg tggatcccag ccagggatgc aagaatctga ctttctcaaa cagataacaa |
| 1501 | cagtcgaaga actggaaccg aaagcaaata actgcactaa ggttctcgtg tggcacactc |
| 1561 | ggacagagaa ggttaatcta gccaacgagc caaagtacca cctggacaca gtgaaaattg |
| 1621 | aggtataaat tgaagcagca actggtgcag tttgtccagc cagtggatcc atatggaaga |
| 1681 | ggatgtttgg agtttaggct acagagcatt caggtattgt ttgttttact tcagtacagc |
| 1741 | agcctttctt gtcatctgat ggacatctgt ttaaatggag cttgtcagtt aacataagct |
| 1801 | aattggatgg ttggtacaaa atgtatgttt tgtcttcatt tgttctgcat gttttctcta |
| 1861 | caacaactaa attggaagat ttttttgtac agtgccgata ctgcaagata ccactcttga |
| 1921 | gtata |

(SEQ ID NO:3) (Homo sapiens beta-1,3-glucuronyltransferase 2 (glucuronosyltransferase S) (B3GAT2), mRNA NCBI Reference Sequence: NM_{—}080742.2), which encodes a protein having an amino acid sequence

MKSALFTRFFILLPWILIVIIMLDVDTRRPVPPLTPRPYFSPYAVGRGGA
RLPLRRGGPAHGTQKRNQSRPQPQPEPQLPTIYAITPTYSRPVQKAELTR
LANTFRQVAQLHWILVEDAAARSELVSRFLARAGLPSTHLHVPTPRRYKR
PGLPRATEQRNAGLAWLRQRHQHQRAQPGVLFFADDDNTYSLELFQEMRT
TRKVSVWPVGLVGGRRYERPLVENGKVVGWYTGWRADRPFAIDMAGFAVS
LQVILSNPKAVFKRRGSQPGMQESDFLKQITTVEELEPKANNCTKVLVWH
TRTEKVNLANEPKYHLDTVKIEV

### (SEQ ID NO:4).

2. GLcATp

In some embodiments, the methods of inhibiting or reducing the expression or production of SGPG include inhibiting or reducing expression of a Glucuronosyl transferase-P. Glucuronosyltransferase-P (GLcATp), also known as B3GAT1 (Beta-1,3-glucuronyltransferase 1) is expressed mainly in the brain. GLcATp functions as the key enzyme in a glucuronyl transfer reaction during the biosynthesis of the carbohydrate epitope HNK-1.

The coding sequence of GLcATp can be

| | |
|---|---|
| 1 | ggagggagcc gcacgcggcc cagggcagcg gtctaggggc gccggggccg gggcgtaggg |
| 61 | gccgttgccc gcgatggacc gcaccggaga cgctccggac tcgtcgccgc aggtgtccac |
| 121 | cccccagggt tcctgacccc tgcccctgga cagcgacccc ttctcagact ccagttgggc |
| 181 | cggactctcc aaacctgctt ccgcaatggg tgggttgtga gtgctggtaa tgaggagccg |
| 241 | tgggtgcagc cagccttgga gatgccgaag agacgggaca tcctagcgat cgtcctcatc |
| 301 | gtgctgccct ggactctgct catcactgtc tggcaccaga gcaccctcgc acccctgctc |
| 361 | gcggtacata aggatgaggg cagtgacccc cgacgcgaaa cgccgcccgg cgccgacccc |
| 421 | agggagtact gcacgtctga ccgcgacatc gtggaggtgg tgcgcaccga gtacgtgtac |
| 481 | acgcggcccc cgccatggtc cgacacgctg cccaccatcc acgtggtgac gcccacctac |
| 541 | agccgcccgg tgcagaaggc cgagctgacg cgcatggcca acacgctgct gcacgtgccc |
| 601 | aacctccact ggctggtggt ggaggatgcg ccgcgccgga cgccgctgac cgcgcgcctg |
| 661 | ctgcgcgaca ccggcctcaa ctacacgcac ctgcacgtgg agacgccccg caactacaag |
| 721 | ctgcgcggag acgcccgcga cccacgcatc ccgcggggca ccatgcagcg caacctggcc |
| 781 | ctgcgctggc tgcgcgagac cttcccgcgc aactccagcc agcctggcgt ggtctacttc |
| 841 | gccgacgacg acaacaccta cagcctggag ctcttcgaag agatgcgcag caccaggagg |
| 901 | gtgtccgtgt ggcccgtcgc cttcgtgggt ggcctgcggt acgaggcccc acgggtgaac |
| 961 | ggggcaggga aggtggtcgg ctggaagacg gtgtttgacc cccaccggcc atttgcaata |
| 1021 | gacatggctg gatttgccgt caacctgcgg ctcattctgc agcgaagcca ggcctacttc |
| 1081 | aagctgcgag gtgtgaaggg aggctaccag gaaagcagcc tccttcgaga acttgtcacc |
| 1141 | ctcaacgacc tggagcccaa ggcagccaac tgcaccaaga tcctggtgtg gcacacacgg |
| 1201 | acagagaagc cagtgctggt gaatgagggc aagaagggct tcactgaccc ctcggtggag |
| 1261 | atctgagcct caggatgcag gagcctcctc ctcagaccct gttcttggcc ttccatcctc |
| 1321 | tccccacggc tgatggtccc tccaaggccg actcctaagg aatcaccatc accctccttt |
| 1381 | ctattctggg ggcttctgag agagcccagc ctgatgccag aacaaaggac agagaattta |
| 1441 | agcacagaaa tcccagacct gttgttctct ccatccagcg tgaccagggc ccgagagacc |
| 1501 | tgatggccag ggtggggtgt ccagcaccag ccaagctggt gctccagcgc acctccccag |
| 1561 | agctccccgc actgacgggg ctgcaggagc aggtgcagtg ggcgcccaca ctggccctgc |
| 1621 | agtgatgcag ggcgggaggg agataagaag accccgcagt caagtggagc atggccctcc |
| 1681 | ctggctccct gtccctgggc tcagcacgac cacacaggac acccagccag ggaattctga |
| 1741 | agaccagaga gcagcccacg ggcatcacga gcgctctgct cctctcctgg gcccctgctc |
| 1801 | ttcccgagag ctgcccccaa atcagacata cctctgtggc tctcctctgg ttcacgttta |
| 1861 | cagagcataa ggctgtcttg gatcccaaca ggcacccagc cctgcatggg gggagcctgg |
| 1921 | gcctaatagg caccccctgt acctcaggct gtggcgggag cagagtcccc ccctccggcc |
| 1981 | cctcttcctt taccccttct cctccagcag tggcaaaggg gtaggctcta gagccagcac |
| 2041 | aggtcactgc ctgacctgga ctaagaaccc cacggcccca ctgtccacac actgcctccc |
| 2101 | caccgcccac ctcggctgct aggcccctcg cctggactgg actggggagg gaaagcgcct |
| 2161 | tttcctgcag ctcttcagag ccacagacct cagggtggag tgagcccatg gtgggcagtg |
| 2221 | ggcaaggcgg tgggtggtgg gcaaggtggg acctcctgca gcctggaaag aggagggagg |
| 2281 | ccaaggccat tccctaactc cctcctgccc ctggtctgag gaggagggac tctggagtag |
| 2341 | cagaggggct gggaaagagg gggcaggggc tgctgggaca ctgagcagga gggaggcctg |
| 2401 | agcacactgc tttggaaatt attctaaaca caaaaaaggg aaagaaaatg ttatttctcc |
| 2461 | ctaagtcagg agcatgcaga gctagcccac ctcatgtcca gctgtccact ttccatcctg |
| 2521 | gagaaagaac agtgtgcctc aaactcctgc cctccccagg cctctggggc ccactggaaa |
| 2581 | gggctctgac cccctggccc agccgggctc tctagtggtg atccggctca ttctcctgca |
| 2641 | agttggaagc acaattttcc ccccaagtgg aggaaaagga aagggcccca gcctactgaa |
| 2701 | gaggtgttta ttttttaact aacagcctcc caccccatta agactcacca ggagaggtct |
| 2761 | gagggccatt cagaacccac tcctgagtgg gtgggtgggt gggactcagt ccagagacct |
| 2821 | aacattcaga atatagcatt ggttgcctat tttgagatgg atttaatctc ccacagtatt |
| 2881 | catgagacca tctgatggaa tcagatccct gagccacctt gcaggacgtt ttccccaacc |
| 2941 | tcttacaccc tggatgtcac tttggaaacc aagcccttgg aagcaagtgg ggtggcatgg |
| 3001 | gagagaaggg aggaggtggg cacaggtggt gagcttatgt gtgggcactc tactgcctca |
| 3061 | cagaagccag ccaagtgcca aggtcagctt ggctggtctg aggccacctt cttagccaaa |
| 3121 | aacctagggt tcattttcag gactttgata atgaacaaca aaatggggac ttctttgggc |
| 3181 | agatgctagg tcagttgttt tcacctaata tcctctttta gctgcatgta tatttattta |
| 3241 | taattataac cctggtggac tgcagccttc atctttattg ggaatgagtt tgttataaat |
| 3301 | cagaaatggg tccatgatga ccactgtttt ccaaacccag tctgttccct gctccctcgc |
| 3361 | tggcaagccc caccacacag gagtgaggcc aggggctagg agttctaaga acagaggctg |
| 3421 | gggtgagggt ggcacccagg cagctgcatc tggtctgttt taatttaact gtatttaatt |
| 3481 | tgctttcaaa attaaaagtc aaatacagtt tttaacagtc ctaaaaaaaa aaaa |

(SEQ ID NO:5); (Homo sapiens beta-1,3-glucuronyltransferase 1 (glucuronosyltransferase P) (B3GAT1), transcript variant 1, mRNA NCBI Reference Sequence: NM_{—}018644.3);
or

| | |
|---|---|
| 1 | ggagggagcc gcacgcggcc cagggcagcg gtctaggggc gccggggccg gggcgtaggg |
| 61 | gccgttgccc gcgatggacc gcaccggaga cgctccggac tcgtcgccgc aggtgtccac |
| 121 | cccccagggt tcctgacccc tgcccctgga cagcgacccc ttctcagact ccagttgggc |
| 181 | cggactctcc aaacctgctt ccgcaatggg tgggttgtga gtgctggtaa gacctgctag |
| 241 | ccaacattca gctgctctgt cctctccatg cctggccggc ccggcccatg cctgttcttt |
| 301 | tctcccctgt gctgccgccg cccgtggccg cccctctcct gaacttaccg ccactcaggt |
| 361 | aatgaggagc cgtgggtgca gccagccttg gagatgccga agagacggga catcctagcg |
| 421 | atcgtcctca tcgtgctgcc ctggactctg ctcatcactg tctggcacca gagcaccctc |
| 481 | gcacccctgc tcgcggtaca taaggatgag ggcagtgacc cccgacgcga aacgccgccc |
| 541 | ggcgccgacc ccagggagta ctgcacgtct gaccgcgaca tcgtggaggt ggtgcgcacc |
| 601 | gagtacgtgt acacgcggcc cccgccatgg tccgacacgc tgcccaccat ccacgtggtg |
| 661 | acgcccacct acagccgccc ggtgcagaag gccgagctga cgcgcatggc caacacgctg |
| 721 | ctgcacgtgc ccaacctcca ctggctggtg gtggaggatg cgccgcgccg gacgccgctg |
| 781 | accgcgcgcc tgctgcgcga caccggcctc aactacacgc acctgcacgt ggagacgccc |
| 841 | cgcaactaca agctgcgcgg agacgcccgc gacccacgca tcccgcgggg caccatgcag |
| 901 | cgcaacctgg ccctgcgctg gctgcgcgag accttcccgc gcaactccag ccagcctggc |
| 961 | gtggtctact tcgccgacga cgacaacacc tacagcctgg agctcttcga agagatgcgc |
| 1021 | agcaccagga gggtgtccgt gtggcccgtc gccttcgtgg gtggcctgcg gtacgaggcc |
| 1081 | ccacgggtga acggggcagg gaaggtggtc ggctggaaga cggtgtttga cccccaccgg |
| 1141 | ccatttgcaa tagacatggc tggatttgcc gtcaacctgc ggctcattct gcagcgaagc |
| 1201 | caggcctact tcaagctgcg aggtgtgaag ggaggctacc aggaaagcag cctccttcga |
| 1261 | gaacttgtca ccctcaacga cctggagccc aaggcagcca actgcaccaa gatcctggtg |
| 1321 | tggcacacac ggacagagaa gccagtgctg gtgaatgagg gcaagaaggg cttcactgac |
| 1381 | ccctcggtgg agatctgagc ctcaggatgc aggagcctcc tcctcagacc ctgttcttgg |
| 1441 | ccttccatcc tctccccacg gctgatggtc cctccaaggc cgactcctaa ggaatcacca |
| 1501 | tcaccctcct ttctattctg ggggcttctg agagagccca gcctgatgcc agaacaaagg |
| 1561 | acagagaatt taagcacaga aatcccagac ctgttgttct ctccatccag cgtgaccagg |
| 1621 | gcccgagaga cctgatggcc agggtggggt gtccagcacc agccaagctg gtgctccagc |
| 1681 | gcacctcccc agagctcccc gcactgacgg ggctgcagga gcaggtgcag tgggcgccca |
| 1741 | cactggccct gcagtgatgc agggcgggag ggagataaga agaccccgca gtcaagtgga |
| 1801 | gcatggccct ccctggctcc ctgtccctgg gctcagcacg accacacagg acacccagcc |
| 1861 | agggaattct gaagaccaga gagcagccca cgggcatcac gagcgctctg ctcctctcct |
| 1921 | gggcccctgc tcttcccgag agctgccccc aaatcagaca tacctctgtg gctctcctct |
| 1981 | ggttcacgtt tacagagcat aaggctgtct tggatcccaa caggcaccca gccctgcatg |
| 2041 | gggggagcct gggcctaata ggcaccccct gtacctcagg ctgtggcggg agcagagtcc |
| 2101 | ccccctccgg cccctcttcc tttacccctt ctcctccagc agtggcaaag gggtaggctc |
| 2161 | tagagccagc acaggtcact gcctgacctg gactaagaac cccacggccc cactgtccac |
| 2221 | acactgcctc cccaccgccc acctcggctg ctaggcccct cgcctggact ggactgggga |
| 2281 | gggaaagcgc cttttcctgc agctcttcag agccacagac ctcagggtgg agtgagccca |
| 2341 | tggtgggcag tgggcaaggc ggtgggtggt gggcaaggtg ggacctcctg cagcctggaa |
| 2401 | agaggaggga ggccaaggcc attccctaac tccctcctgc ccctggtctg aggaggaggg |
| 2461 | actctggagt agcagagggg ctgggaaaga gggggcaggg gctgctggga cactgagcag |
| 2521 | gagggaggcc tgagcacact gctttggaaa ttattctaaa cacaaaaaag ggaaagaaaa |
| 2581 | tgttatttct ccctaagtca ggagcatgca gagctagccc acctcatgtc cagctgtcca |
| 2641 | ctttccatcc tggagaaaga acagtgtgcc tcaaactcct gccctcccca ggcctctggg |
| 2701 | gcccactgga aagggctctg accccctggc ccagccgggc tctctagtgg tgatccggct |
| 2761 | cattctcctg caagttggaa gcacaatttt ccccccaagt ggaggaaaag gaaagggccc |
| 2821 | cagcctactg aagaggtgtt tattttttaa ctaacagcct cccaccccat taagactcac |
| 2881 | caggagaggt ctgagggcca ttcagaaccc actcctgagt gggtgggtgg gtgggactca |
| 2941 | gtccagagac ctaacattca gaatatagca ttggttgcct attttgagat ggatttaatc |
| 3001 | tcccacagta ttcatgagac catctgatgg aatcagatcc ctgagccacc ttgcaggacg |
| 3061 | ttttccccaa cctcttacac cctggatgtc actttggaaa ccaagccctt ggaagcaagt |
| 3121 | ggggtggcat gggagagaag ggaggaggtg ggcacaggtg gtgagcttat gtgtgggcac |
| 3181 | tctactgcct cacagaagcc agccaagtgc caaggtcagc ttggctggtc tgaggccacc |
| 3241 | ttcttagcca aaaacctagg gttcattttc aggactttga taatgaacaa caaaatgggg |
| 3301 | acttctttgg gcagatgcta ggtcagttgt tttcacctaa tatcctcttt tagctgcatg |
| 3361 | tatatttatt tataattata accctggtgg actgcagcct tcatctttat tgggaatgag |
| 3421 | tttgttataa atcagaaatg ggtccatgat gaccactgtt ttccaaaccc agtctgttcc |
| 3481 | ctgctccctc gctggcaagc cccaccacac aggagtgagg ccaggggcta ggagttctaa |
| 3541 | gaacagaggc tggggtgagg gtggcaccca ggcagctgca tctggtctgt tttaatttaa |
| 3601 | ctgtatttaa tttgctttca aaattaaaag tcaaatacag tttttaacag tcctaaaaaa |
| 3661 | aaaaaa |

(SEQ ID NO:6) (Homo sapiens beta-1,3-glucuronyltransferase 1 (glucuronosyltransferase P) (B3GAT1), transcript variant 2, mRNA NCBI Reference Sequence: NM_{—}054025.2) each of which encode a protein having the amino acid sequence:

MPKRRDILAIVLIVLPWTLLITVWHQSTLAPLLAVHKDEGSDPRRETPPG
ADPREYCTSDRDIVEVVRTEYVYTRPPPWSDTLPTIHVVTPTYSRPVQKA
ELTRMANTLLHVPNLHWLVVEDAPRRTPLTARLLRDTGLNYTHLHVETPR
NYKLRGDARDPRIPRGTMQRNLALRWLRETFPRNSSQPGVVYFADDDNTY
SLELFEEMRSTRRVSVWPVAFVGGLRYEAPRVNGAGKVVGWKTVFDPHRP
FAIDMAGFAVNLRLILQRSQAYFKLRGVKGGYQESSLLRELVTLNDLEPK
AANCTKILVWHTRTEKPVLVNEGKKGFTDPSVEI

### (SEQ ID NO:7).

### III. Compositions for Use in Methods of Maintaining Blood-Brain Barrier and Blood-Nerve Integrity

Compositions for use in the disclosed methods of maintaining blood-brain barrier and blood-nerve integrity are provided. Typically the compositions include an antagonist of glucuronoslytransferase antagonist, an antagonist of killer epitope-1 sulfotransferase (HNK-1ST), or a combination thereof.

In some in vivo approaches, the compositions are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. For example, the antagonist can be provided in an effective amount to reduce expression of sulfated glucuronosyl paragloboside (SGPG) in the subject and thereby reduce apoptosis of endothelial cells of the blood-brain barrier or the blood-nerve barrier in the subject. The precise dosage will vary according to a variety of factors such as subject-dependent variables (e.g., age, immune system health, etc.), the disease, and the treatment being effected.

In some embodiments, the reduction in expression of the target molecule, sulfated glucuronosyl paragloboside (SGPG), or apoptosis of endothelial cells in a subject treated with the antagonist is relative to a control. Suitable controls are known in the art and can be, for example, a subject that has not been treated with the antagonist.

In preferred embodiments, the composition has controlled or time-limited effect on the subject. Accordingly, in a preferred embodiment, the composition does not cause a permenant of irreversible change in glucuronoslytransferase or HNK-1ST expression in the subject.

A. Antagnoists

Glucuronoslytransferase antagonists and antagonists of killer epitope-1 sulfotransferase (HNK-1ST) are provided.

1. Functional Nucleic Acids

In some embodiments, the antagonist is a function nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include, but are not limited to, antisense molecules, siRNA, miRNA, aptamers, ribozymes, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the polypeptide itself. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹².

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10-12 M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a K_{d} with the target molecule at least 10, 100, 1000, 10,000, or 100,000 fold lower than the K_{d} with a background binding molecule. It is preferred when doing the comparison for a polypeptide for example, that the background molecule be a different polypeptide.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹².

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukarotic cells. Representative examples of how to make and use EGS molecules to facilitate cleavage of a variety of different target molecules are known in the art.

Gene expression can also be effectively silenced in a highly specific manner through RNA interference (RNAi). This silencing was originally observed with the addition of double stranded RNA (dsRNA) (Fire, A., et al., Nature, 391:806-11 (1998); Napoli, C., et al., Plant Cell, 2:279-89 (1990); Hannon, G. J., Nature, 418:244-51 (2002)). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, S. M., et al., Genes Dev., 15:188-200 (2001); Bernstein, E., et al. Nature, 409:363-6 (2001); Hammond, S. M., et al., Nature, 404:293-6 (2000)). In an ATP dependent step, the siRNAs become integrated into a multi-subunit protein complex, commonly known as the RNAi induced silencing complex (RISC), which guides the siRNAs to the target RNA sequence (Nykanen, A., et al., Cell, 107:309-21 (2001)). At some point the siRNA duplex unwinds, and it appears that the antisense strand remains bound to RISC and directs degradation of the complementary mRNA sequence by a combination of endo and exonucleases (Martinez, J., et al., Cell, 110:563-74 (2002)). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al., Nature, 411:494 498 (2001); Ui-Tei, K., et al., FEBS Lett. 479:79-82 (2000)). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-ITT™ inducible RNAi plasmid and lentivirus vectors. Disclosed herein are any shRNA designed as described above based on the sequences for the herein disclosed transferases.

Therefore, in some embodiment the antagonist is a functional nucleic acid designed to target a glucuronosyltransferase or killer epitope-1 sulfotransferase (HNK-1ST). For example, antisense oligonucleotides, RNAi, dsRNA, miRNA, siRNA, external guide sequences, and the like can be designed to target a glucuronosyltransferase or killer epitope-1 sulfotransferase.

In some embodiments, the antisense oligonucleotide, RNAi, dsRNA, miRNA, siRNA, external guide sequence is designed to target a HNK-1ST that can reduce or inhibit expression of the nucleic acid sequence of SEQ ID NO:1, or variant thereof having 70%, 75%, 80%, 85%, 90%, 95%, 97%, 99% or more sequence identity to SEQ ID NO:1, or a nucleic acid encoding the amino acid sequence of SEQ ID NO:2.

For example, siRNA designed to target HNK-1ST mRNA can be prepared using the primer sequences: Duplex 5: sense, GCU GAU UGU UCU AAA UGG AUU (SEQ ID NO:8) and anti-sense, 5′-P UCC AUU UAG AAC AAU CAG CUU (SEQ ID NO:9); Duplex 6: sense, GUA AGA GAU CCC UUC GAA AUU (SEQ ID NO:10) and anti-sense: 5′-P UUU CGA AGG GAU CUC UUA CUU (SEQ ID NO:11); Duplex 7: sense, UGA CAA CCA UGC CGG AGG UUU (SEQ ID NO:12) and anti-sense, 5′-P ACU UCC GGC AUG GUU GUC AUU (SEQ ID NO:13); Duplex 8: sense, CUA GCA AGU UCA UCA CGU UUU (SEQ ID NO:14) and anti-sense, 5′-P AAC GUG AUG AAC UUG CUA GUU (SEQ ID NO:15).

In some embodiments, the antisense oligonucleotide, RNAi, dsRNA, miRNA, siRNA, external guide sequence is designed to target a HNK-1ST that can reduce or inhibit expression of the nucleic acid sequence of SEQ ID NO:3, 5, or 6, or variant thereof having 70%, 75%, 80%, 85%, 90%, 95%, 97%, 99% or more sequence identity to SEQ ID NO:3, 5, or 6, or a nucleic acid encoding the amino acid sequence of SEQ ID NO:7.

As discussed above, in some embodiments, is preferred that administration of the antagonist does not induce a permanent or non-reversible change glucuronoslytransferase or HNK-1ST expression. Onset, maintenance and extinction of behavioral effects of antisense oligonucleotides are dependent on time after application. A number of studies have reported the recovery of behavioral effects after the termination of antisense olignucleotide treatment, suggesting that the blockade of gene expression and function are reversible. For example, inhibition of 5 receptor agonist-mediated analgesia by antisense olignucleotide administered intrathecally three times every other day (days 1, 3 and 5) was greatest on day 6 (−80% compared with four bases-mismatchd olignucleotide or vehicle) but had recovered by 5 days after the last injection (day 10). Therefore, in some embodiments, administration and expression of antisense oligonucleotides, RNAi, dsRNA, miRNA, siRNA, external guide sequence, such as those describe above is transient.

2. Nucleic Acid Variants and Derivatives

The disclosed nucleic acids can be made up of for example, nucleotides, nucleotide analogs, or nucleotide substitutes. Non-limiting examples of these and other molecules are discussed herein. It is understood that for example, the disclosed antisense nucleic acid sequences will typically be made up of A, C, G, and U/T. Likewise, it is understood that if a nucleic acid molecule is introduced into a cell or cell environment through for example exogenous delivery, it is advantageous that the nucleic acid molecule be made up of nucleotide analogs that reduce the degradation of the nucleic acid molecule in the cellular environment.

So long as their relevant function is maintained, the disclosed nucleic acids can be made up of or include modified nucleotides (nucleotide analogs). Many modified nucleotides are known and can be used in oligonucleotides and nucleic acids. A nucleotide analog is a nucleotide which contains some type of modification to the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine, and 5-methylcytosine can increase the stability of duplex formation.

Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve unique properties such as increased duplex stability. Compositions and method for base modifications, their synthesis, their use, and their incorporation into oligonucleotides and nucleic acids are known in the art.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10, alkyl or C₂ to C10 alkenyl and alkynyl. 2′ sugar modifications also include but are not limited to —O[(CH₂)nO]mCH₃, —O(CH₂)nOCH₃, —O(CH₂)nNH₂, —O(CH₂)nCH₃, —O(CH₂)n-ONH₂, and —O(CH₂)nON[(CH₂)nCH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications can also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S, Nucleotide sugar analogs can also have sugar mimetics such as cyclobutyl moieties in place of the pentofuranosyl sugar. Methods for preparation of such modified sugar structures, their synthesis, their use, and their incorporation into nucleotides, oligonucleotides and nucleic acids are known in the art.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Methods of making and using nucleotides containing modified phosphates their synthesis, their use, and their incorporation into nucleotides, oligonucleotides and nucleic acids are known in the art.

It is understood that nucleotide analogs need only contain a single modification, but can also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Compositions and methods for making and using these types of phosphate replacements their synthesis, their use, and their incorporation into nucleotides, oligonucleotides and nucleic acids are known in the art.

It is also understood in a nucleotide substitute that both the sugar and the phosphate moieties of the nucleotide can be replaced, by for example an amide type linkage (aminoethylglycine) (PNA). Methods of making and using PNA are known in the art.

Oligonucleotides and nucleic acids can be comprised of nucleotides and can be made up of different types of nucleotides or the same type of nucleotides. For example, one or more of the nucleotides in an oligonucleotide can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. Such oligonucleotides and nucleic acids can be referred to as chimeric oligonucleotides and chimeric nucleic acids.

2. Small Molecules

The term “small molecule” generally refers to small organic compounds having a molecular weight of more than about 100 and less than about 2,500 Daltons, preferably between 100 and 2000, more preferably between about 100 and about 1250, more preferably between about 100 and about 1000, more preferably between about 100 and about 750, more preferably between about 200 and about 500 Daltons. The small molecules can include cyclical carbon or heterocyclic structures and/or aromatic or polyaromatic structures substituted with one or more functional groups. The small molecule antagonist reduces or interferes with expression or production of an SGGL, for example SGPG, or SGLPG. In a preferred embodiment, the small molecule reduces or interferes with expression or function of a glucuronosyltransferase or HNK-1ST.

B. Pharmaceutical Compositions

The disclosed compositions can be employed for therapeutic uses in combination with a suitable pharmaceutical carrier. The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids.

Pharmaceutical compositions including nucleic acids and small molecules are provided. Pharmaceutical compositions can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

For the nucleic acids, small molecules or combinations thereof, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally dosage levels of 0.001 to 10 mg/kg of body weight daily are administered to mammals. Generally, for intravenous injection or infusion, dosage may be lower.

In certain embodiments, the compositions are administered locally, for example by injection directly into a site to be treated. Typically, local injection causes an increased localized concentration of the compositions which is greater than that which can be achieved by systemic administration.

It is understood by one of ordinary skill in the art that nucleotides administered in vivo are taken up and distributed to cells and tissues (Huang, et al., FEBS Lett., 558(1-3):69-73 (2004)). For example, Nyce, et al. have shown that antisense oligodeoxynucleotides (ODNs) when inhaled bind to endogenous surfactant (a lipid produced by lung cells) and are taken up by lung cells without a need for additional carrier lipids (Nyce, et al., Nature, 385:721-725 (1997)). Small nucleic acids are readily taken up into T24 bladder carcinoma tissue culture cells (Ma, et al., Antisense Nucleic Acid Drug Dev., 8:415-426 (1998)).

The disclosed compositions including olignucleotides may be in a formulation for administration topically, locally or systemically in a suitable pharmaceutical carrier. Remington, The Science and Practice of Pharmacy, 22nd edition, (Edited by Allen, Loyd V., Jr), Pharmaceutical Press (2012), discloses typical carriers and methods of preparation. The compound may also be encapsulated in suitable biocompatible microcapsules, microparticles, nanoparticles, or microspheres formed of biodegradable or non-biodegradable polymers or proteins or liposomes for targeting to cells. Such systems are well known to those skilled in the art and may be optimized for use with the appropriate nucleic acid.

For example, in general, the disclosed compositions can be incorporated within or on microparticles. As used herein, microparticles include liposomes, virosomes, microspheres and microcapsules formed of synthetic and/or natural polymers. Methods for making microcapsules and microspheres are known to those skilled in the art and include solvent evaporation, solvent casting, spray drying and solvent extension. Examples of useful polymers which can be incorporated into various microparticles include polyesters, polysaccharides, polyanhydrides, polyorthoesters, polyhydroxides and proteins and peptides.

Liposomes can be produced by standard methods such as those reported by Kim, et al., Biochim. Biophys. Acta, 728, 339-348 (1983); Liu, D., et al., Biochim. Biophys. Acta, 1104, 95-101 (1992); and Lee, et al., Biochim. Biophys. Acta., 1103, 185-197 (1992); Wang, et al., Biochem., 28, 9508-9514 (1989)), incorporated herein by reference. The disclosed compositions can be encapsulated within liposomes when the molecules are present during the preparation of the microparticles. Briefly, the lipids of choice, dissolved in an organic solvent, are mixed and dried onto the bottom of a glass tube under vacuum. The lipid film is rehydrated using an aqueous buffered solution of the composition to be encapsulated, and the resulting hydrated lipid vesicles or liposomes encapsulating the material can then be washed by centrifugation and can be filtered and stored at 4° C. This method has been used to deliver nucleic acid molecules to the nucleus and cytoplasm of cells of the MOLT-3 leukemia cell line (Thierry, A. R. et al., Nucl. Acids Res., 20: 5691-5698 (1992)). Alternatively the disclosed compositions can be incorporated within microparticles, or bound to the outside of the microparticles, either ionically or covalently.

Cationic liposomes or microcapsules are microparticles that are particularly useful for delivering negatively charged compounds such as nucleic acid-based compounds, which can bind ionically to the positively charged outer surface of these liposomes. Various cationic liposomes have previously been shown to be very effective at delivering nucleic acids or nucleic acid-protein complexes to cells both in vitro and in vivo, as reported by Felgner, P. L. et al., Proc. Natl. Acad. Sci. USA, 84: 7413-7417 (1987); Felgner, P. L., Advanced Drug Delivery Reviews, 5: 163-187 (1990); Clarenc, J. P. et al., Anti-Cancer Drug Design, 8: 81-94 (1993). Cationic liposomes or microcapsules can be prepared using mixtures including one or more lipids containing a cationic side group in a sufficient quantity such that the liposomes or microcapsules formed from the mixture possess a net positive charge which will ionically bind negatively charged compounds. Examples of positively charged lipids that may be used to produce cationic liposomes include the aminolipid dioleoyl phosphatidyl ethanolamine (PE), which possesses a positively charged primary amino head group; phosphatidylcholine (PC), which possess positively charged head groups that are not primary amines; and N[1-(2,3-dioleyloxy)propyl]N,N,N-triethylammonium (“DOTMA,” see Feigner, P. L. et al., Proc. Natl. Acad. Sci. USA, 84, 7413-7417 (1987); Feigner, P. L. et al., Nature, 337, 387-388 (1989); Feigner, P. L., Advanced Drug Delivery Reviews, 5, 163-187 (1990)).

Nucleic acid can also be encapsulated by or coated on cationic liposomes which can be injected intravenously into a mammal. This system has been used to introduce DNA into the cells of multiple tissues of adult mice, including endothelium and bone marrow, where hematopoietic cells reside (see, for example, Zhu et al., Science, 261: 209-211 (1993)).

Liposomes containing the nucleic acids, can be administered systemically, for example, by intravenous or intraperitoneal or pulmonary administration, in an amount effective for delivery of the disclosed compositions to targeted cells. Other possible routes include trans-dermal or oral, when used in conjunction with appropriate microparticles. Generally, the total amount of the liposome-associated nucleic acid administered to an individual will be less than the amount of the unassociated nucleic acid that must be administered for the same desired or intended effect.

Compositions including various polymers such as the polylactic acid and polyglycolic acid copolymers, polyethylene, and polyorthoesters and the disclosed compositions can be delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can effect a sustained release of the nucleic acid.

Various methods for nucleic acid delivery are described, for example, in Sambrook and Russell, Molecular Cloning: A Laboratory Manual, 3rd edition (2001); Current Protocols In Molecular Biology [(F. M. Ausubel, et al. eds., (1987)]; Coligan, Dunn, Ploegh, Speicher and Wingfeld, eds. (1995) Current Protocols in Protein Science (John Wiley & Sons, Inc.); the series Methods in Enzymology (Academic Press, Inc.): PCR 2: A Practical Approach (M. J. MacPherson, B. D. Hames and G. R. Taylor eds. (1995)]. Such nucleic acid delivery systems include the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition, as discussed above. The nucleic acid delivery system can be provided to the cell either directly, such as by contacting it with the cell, or indirectly, such as through the action of any biological process. The nucleic acid delivery system can be provided to the cell by endocytosis, receptor targeting, coupling with native or synthetic cell membrane fragments, physical means such as electroporation, combining the nucleic acid delivery system with a polymeric carrier such as a controlled release film or nanoparticle or microparticle, using a vector, injecting the nucleic acid delivery system into a tissue or fluid surrounding the cell, simple diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

Formulations for topical administration may include ointments, lotions, creams, gels, drops, sprays, liquids and powders. Conventional pharmaceutical carriers can be used as desired. Formulations suitable for parenteral administration, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, optionally with an added preservative.

The compositions may take such forms as sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, and electrolyte replenishers (such as those based on Ringer's dextrose). Preservatives and other additives may also be present such as, for example, antimicrobials, antioxidants, chelating agents and inert gases.

In addition, sterile, fixed oils are conventionally employed as a solvent or suspending medium. For this purpose any bland fixed oil including synthetic mono- or di-glycerides may be employed. In addition, fatty acids such as oleic acid may be used in the preparation of injectables. Carrier formulation can be found in Remington's (surpa).

The compositions alone or in combination with other suitable components, can also be made into aerosol formulations (i.e., they can be “nebulized”) to be administered via inhalation. For pulmonary administration, formulations can be administered using a metered dose inhaler (“MDI”), a nebulizer, an aerosolizer, or a dry powder inhaler. Suitable devices are commercially available and described in the literature.

Inhaled aerosols have been used for the treatment of local lung disorders including asthma and cystic fibrosis (Anderson, et al., Am. Rev. Respir. Dis., 140: 1317-1324 (1989)) and have potential for the systemic delivery of peptides and proteins as well (Patton, et al., Advanced Drug Delivery Reviews, 8:179-196 (1992)). Considerable attention has been devoted to the design of therapeutic aerosol inhalers to improve the efficiency of inhalation therapies. Timsina, et. al., Int. J. Pharm., 101: 1-13 (1995); and Tansey, I. P., Spray Technol. Market, 4: 26-29 (1994).

The formulation may be administered alone or in any appropriate pharmaceutical carrier for administration to the respiratory system. Delivery is achieved by one of several methods. For example, the patient can mix a dried powder of oligonucleotide with solvent and then nebulize it. It may be more appropriate to use a pre-nebulized solution, regulating the dosage administered and avoiding possible loss of suspension. After nebulization, it may be possible to pressurize the aerosol and have it administered through a metered dose inhaler (MDI). Nebulizers create a fine mist from a solution or suspension, which is inhaled by the patient. Dry powders are particularly preferred.

Systemic administration can also be by transmucosal means. For transmucosal administration, penetrants appropriate to the barrier to be permeated are used in the formulation. Such penetrants are generally known in the art, and include, for example, for transmucosal administration, detergents, bile salts, and fusidic acid derivatives.

In one embodiment, the oligonucleotides are conjugated to lipophilic groups like cholesterol and lauric and lithocholic acid derivatives with C32 functionality to improve cellular uptake. For example, cholesterol has been demonstrated to enhance uptake and serum stability of siRNA in vitro (Lorenz, et al., Bioorg. Med. Chem. Lett., 14(19):4975-4977 (2004)) and in vivo (Soutschek, et al., Nature, 432(7014):173-178 (2004)).

In addition, it has been shown that binding of steroid conjugated oligonucleotides to different lipoproteins in the bloodstream, such as LDL, protect integrity and facilitate biodistribution (Rump, et al., Biochem. Pharmacol., 59(11):1407-1416 (2000)). Other groups that can be attached or conjugated to the compound described above to increase cellular uptake, include acridine derivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines. U.S. Pat. No. 6,919,208 to Levy, et al., also describes methods for enhanced delivery. These pharmaceutical formulations may be manufactured in a manner that is itself known, e.g., by means of conventional mixing, dissolving, granulating, levigating, emulsifying, encapsulating, entrapping or lyophilizing processes.

### Method of Preventing Endothelial Cell Apoptosis

Preventing endothelial cell apoptosis can improve the strength of the BBB. For example, endothelial cells line the BBB and increased apoptosis in these cells can weaken the BBB allowing unwanted substances, such as inflammatory cells, into the brain. Therefore, preventing endothelial cell apoptosis can be an important aspect of protecting the BBB and limiting the influx of inflammatory cells into the brain.

Preventing endothelial cell apoptosis can occur by reducing or inhibiting GLcAT expression. It can also occur by altering different proteins in the same pathway. For instance, reducing or inhibiting SGGL expression can also play a role in preventing endothelial cell apoptosis. The antisense GLcAT sequences disclosed herein can be used. Other inhibitory agents such as siRNA specific for GLcAT or SGGL can also be used.

The method can be achieved by administering to a subject a sufficient amount of inhibitory agent, such as a GLcAT antisense sequence, to prevent endothelial cell apoptosis.

### V. Disease to be Treated

The disclosed compositions and methods can be employed therapeutically to treating inflammation, particularly neuroinflammation. As discussed above, increased SGGL expression on endothelial cells lining the BBB is involved in promoting T cell adhesion as well as increasing endothelial cell apoptosis. Neuroinflammation can be caused by inflammatory cells, such as T cells, crossing the BBB and infiltrating the central nervous system. Because SGGL expression is involved in T cell adhesion and a weakening of the BBB, it is possible to alter the pathway leading to SGGL expression in order to treat inflammation. Therefore, reducing or inhibiting GLcAT or HNK-1ST expression can be used as a treatment for neuroinflammation.

The disclosed compositions and methods can also be used to treat one or more symptoms of a disease or disorder associated with neuroinflammation. Neuroinflammatory diseases and disorders include, but are not limited to, multiple sclerosis, bacterial meningitis, ischemia, brain edema, AIDS, Guillian-Barre Syndrome, and Alzheimer's Disease.

### VI. Animal Model

An animal model that overexpresses GLcAT can be used to study neuroinflammation. The constructs and nucleic acid sequences disclosed in the Examples below can be used to produce a transgenic GLcAT animal model. In some instances, both the GLcATs and p forms can be overexpressed and in other instances one form or the other can be overexpressed. The overexpression of GLcAT can be used to study the resulting increase in SGGLs on the surface of endothelial cells. The animal models can be used to examine the role of SGGLs in neuroinflammation and methods for blocking or preventing SGGL neuroinflammatory-related responses.

A transgenic animal model means that the animal contains a transgene. A “transgene” is a nucleic acid sequence that is inserted into a cell and becomes a part of the genome of that cell and its progeny. Such a transgene may be (but is not necessarily) partly or entirely heterologous (e.g., derived from a different species) to the cell. The term “transgene” broadly refers to any nucleic acid that is introduced into an animal's genome, including but not limited to genes or DNA having sequences which are perhaps not normally present in the genome, genes which are present, but not normally transcribed and translated (“expressed”) in a given genome, or any other gene or DNA which one desires to introduce into the genome. This may include genes which may normally be present in the nontransgenic genome but which one desires to have altered in expression, or which one desires to introduce in an altered or variant form or in a different chromosomal location. A transgene can include one or more transcriptional regulatory sequences and any other nucleic acid, such as introns, that may be useful or necessary for optimal expression of a selected nucleic acid. A transgene can be as few as a couple of nucleotides long, but is preferably at least about 50, 100, 150, 200, 250, 300, 350, 400, or 500 nucleotides long or even longer and can be, e.g., an entire genome. A transgene can be coding or non-coding sequences, or a combination thereof. A transgene usually comprises a regulatory element that is capable of driving the expression of one or more transgenes under appropriate conditions. By “transgenic animal” is meant an animal comprising a transgene as described above. Transgenic animals are made by techniques that are well known in the art. The disclosed nucleic acids, in whole or in part, in any combination, can be transgenes as disclosed herein.

The disclosed transgenic animals can be any non-human animal, including a non-human mammal (e.g., mice, rats, rabbits, guinea pigs, pigs, primates, etc. . . . ), bird or an amphibian, in which one or more cells contain heterologous nucleic acid introduced by way of human intervention, such as by transgenic techniques well known in the art.

Generally, the nucleic acid is introduced into the cell, directly or indirectly, by introduction into a precursor of the cell, such as by microinjection or by infection with a recombinant virus. The disclosed transgenic animals can also include the progeny of animals which had been directly manipulated or which were the original animal to receive one or more of the disclosed nucleic acids. The transgene may be integrated within a chromosome, or it may be an extrachromosomal replicating DNA. Techniques related to the production of transgenic animals are known in the art (Hogan, et al., Manipulating the Mouse Embryo—A Laboratory Manual Cold Spring Harbor Laboratory, Cold Spring Harbor, N.Y., 1986).

### EXAMPLES

### Example 1

### Inhibition of the HNK-1ST Gene Reduces NF-κB Activity by Inhibiting IκB Activation in SVHCECs After Cytokine Stimulation but Promoted ERK Activation

### Materials and Methods

Primers

Human TNFα and IL-1β were purchased from PeproTech (Rocky Hill, N.J.), siRNA HNK-1ST SMARTpool was purchased from Dharmacon Inc. (Thermo Fisher Scientific, Lafayette, Colo.), with the following primer sequences: Duplex 5: sense, GCU GAU UGU UCU AAA UGG AUU (SEQ ID NO:8) and anti-sense, 5′-P UCC AUU UAG AAC AAU CAG CUU (SEQ ID NO:9); Duplex 7: sense, GUA AGA GAU CCC UUC GAA AUU (SEQ ID NO:10) and anti-sense: 5′-P UUU CGA AGG GAU CUC UUA CUU (SEQ ID NO:11); Duplex 7: sense, UGA CAA CCA UGC CGG AGG UUU (SEQ ID NO:12) and anti-sense, 5′-P ACU UCC GGC AUG GUU GUC AUU (SEQ ID NO:13); Duplex 8: sense, CUA GCA AGU UCA UCA CGU UUU (SEQ ID NO:14) and anti-sense, 5′-P AAC GUG AUG AAC UUG CUA GUU (SEQ ID NO:15). A scrambled siRNA SMARTpool was used to compare the transfection performance and validity of the results. The siRNA sample was dissolved in 1×siRNA buffer (0.5 ml≡20 μM) and preserved into 10 tubes (50 μl each). The dual luciferase reporter assay system was purchased from Promega (Madison, Wis.). Cell culture media and growth factors were purchased from Invitrogen (Carlsbad, Calif.). Purified SGPG and its monoclonal antibody (NGR 50, mouse IgG) were received as generous gifts from Dr. Toshio Ariga, Institute of Molecular Medicine and Genetics, Georgia Health Sciences University, Augusta, Ga. Antibodies against IKB, ERK, and caspase 3 were purchased from Cell Signaling Technology (Danvers, Mass.). In situ cell detection kit was purchased from Roche Applied Science (Indianapolis, Ind.). Heparin (Na-salt) was obtained from Sigma Chemical (St. Louis, Mo.). All reagents, buffers, and chemicals were of analytical grades.

Cell Culture

Endothelial cells of human cerebro-microvascular origin (SV-HCECs) (Muruganandam et al. 1997) which were generously supplied by Dr. D. Stanimirovic, National Research Council of Canada, Ottawa, Canada, were grown in 0.5% gelatin-coated dish in media (Media 199, Invitrogen) containing insulin-transferrin-selenium, heparin, and penicillinstreptomycin (Cellgro) as described previously (Dasgupta S, et al., J. Neurosci, Res., 85:1086-1094 (2007); Muruganandam A, et al., FASEB J., 11:1187-1197 (1997); Duvar S, et al., J. Neurochem., 75:1970-1976 (2000)).

Inhibition of HNK-1ST Gene Expression by HNK-1ST siRNA Transfection

Cells were transfected in Amaxa Nucleofector equipment using T20 Program in an aseptic condition as described previously (Dasgupta S, et al., J. Neurosci. Res., 87:3591-3599 (2009); Dasgupta S, et al., J. Neurosci. Res., 85:1086-1094 (2007)). Briefly, cells were grown in 3×100-mm dishes, collected by trypsinization, counted, and divided into 3 groups (1.0×106 cells per group). Cells were then suspended in 0.1 ml of transfecting media in the presence of HNK-1ST siRNA (7.5 μl≡150 pmol). Controls were prepared simultaneously using a scrambled siRNA mixture for comparison. The mixture containing the cell suspension was transferred into a sterile cuvette (2-mm gap) and zapped using the T20 Program. Five hundred μl of the pre-warmed medium (complete) was added immediately, and the cells were aspirated carefully. The transfected cells were dispersed in 3×60-mm dish, pre-coated with 0.5% gelatin. The media were changed after 4-6 h of transfection, and cells were incubated for 48 h before being exposed to the inflammatory cytokines. After incubation for another 18-24 h, cells were washed with cold PBS, and total proteins were dissolved in Lamelli buffer for examining cell signaling. Protein content was measured using RcDc reagents (Bio-Rad, Hercules, Calif.). A time-dependent ERK activation was studied using IL-1β (25 ng/ml) and TNFα (100 ng/ml) at 0 h, 2 h, 4 h, 8 h, 12 h and 24 h. The data indicate that both IL-1β and TNFα stimulated ERK optimally at 24 h. Accordingly, the cells were exposed between 18-24 h.

Identification of Signaling Molecules by Western Blot Analysis

A defined amount of the protein (20-30 μg) was applied on a gradient polyacrylamide gel (Bio-Rad, Hercules, Calif.) and subjected to electrophoresis. The protein bands were transferred onto a PVDF membrane and visualized by Ponceau staining. The following specified proteins were identified using Western blot analysis, phospho- and total IKB (for NF-κB activity), phospho- and total ERK, phospho- and total Akt, and active caspase 3 (for apoptosis). Protein loading was normalized using α-tubulin or β-actin. Bands were identified using a chemilumniscence reagent, ECL (GE Health Care, Buckinghamshire, UK). In addition, the activation of two other MAP kinases, phospho-JUN and phospho-P38 were examined for comparison.

### Results

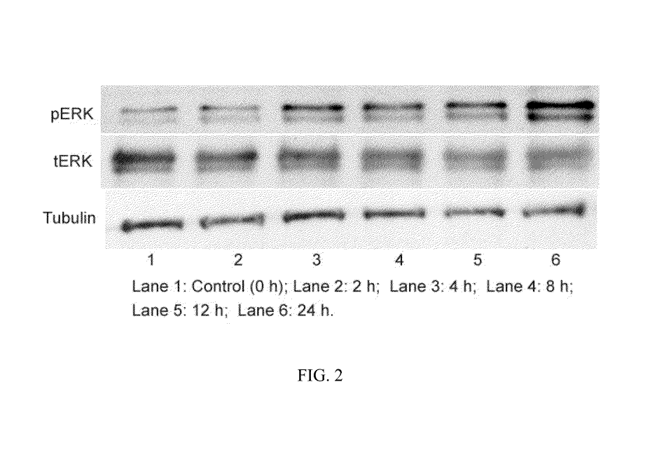

Transfection of cells with HNK-1ST siRNA reduced HNK-1ST gene expression and inhibited SGPG up-regulation by suppressing cytokine-stimulated NF-κB activity (Dasgupta, et al., J. Neurosci. Res., 87(16):3591-9 (2009)). The protein level of IKB and its phosphorylation level were examined to investigate the mechanism. The level of IKB, an inhibitor protein of NF-κB, is controlled by its phosphorylation. Phosphorylated IKB is released and degraded, thus and activating NF-κB (Hayden & Ghosh 2004). Consistent with Dasgupta et al., supra, the IKB protein level was increased by the down-regulation of SGPG through HNK-1ST siRNA and, at the same time the IKB phosphorylation was decreased, with or without cytokine treatment (FIG. 1). It is noteworthy that the time course activation of ERK by IL-1β and TNFα showed an identical profile with optimum activation at 24 h. Hence the study measuring ERK activation using cytokine exposure after siHNK-1 transfection, is in compliance with the time-course study. A representative figure is shown in FIG. 2.

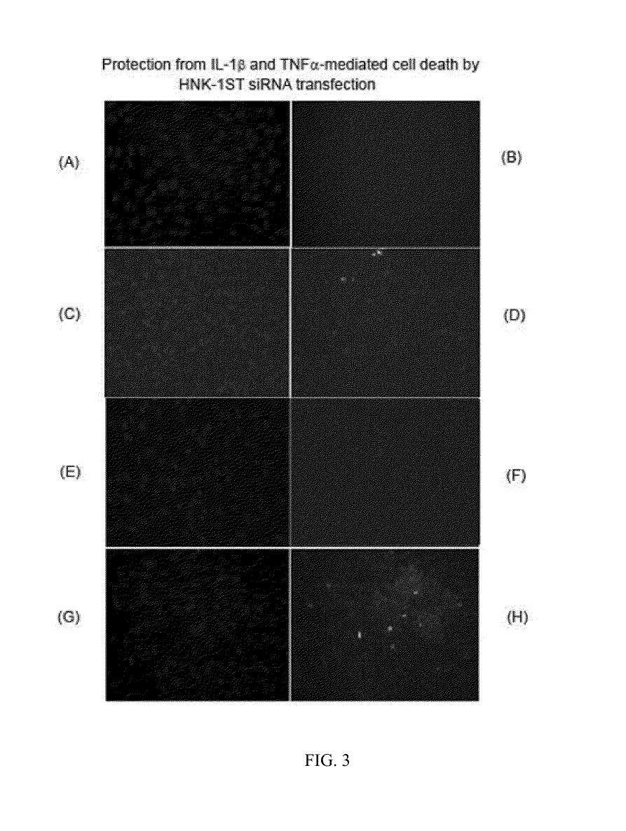

HNK-1ST down-regulation by siRNA rendered the cells more resistant to apoptosis, as measured by reduced caspase 3 activation (FIG. 1) and less TUNEL staining (FIG. 3). Because of the loose attachment of cells on the slide after HNK-1ST siRNA transfection, cytokine exposure time was reduced to 8 h (instead of 24 h). The exposed cells were fixed with PFA, and then subjected to TUNEL staining Approximately 15% cell death was recorded with IL-1β treatment and more than 20-25% cell death after exposure to TNFα.

To investigate the cell survival signaling pathway, Akt and ERK (MAPK) activation were assayed. The data indicated that ERK activation (FIG. 1) could be the factor that protects the cells, leading to enhanced cell survival by SGPG inhibition. Akt and phosphorylated Akt were not affected, however. Activation of two other kinases, JUN and P38 that were not activated by siHNK-1ST transfection, however, P38 kinase showed a mild activation (only 60-80% stimulation) with GATp/GATs transfection.

### Example 2

### GlcATp/GlcATs-Transfection Stimulates SGPG Expression

### Materials and Methods

Construction of EGFP-GlcATp and EGFP-GlcATs Plasmids and Transfection of SV-HCECs with EGFP cDNA Plasmid

For the construction of pcDNA3.1-GlcATp, and GlcATs, human GlcATp and GlcATs cDNA was amplified using following the primers: for GlcATp: sense, 5′-AA CTC GAG ATG CCG AAG AGA CGG GAC ATC CTA G-3′ (Xho-1 site) and antisense, 5′-AA AAG CTT GAT CTC CAC CGA GGG GTC AGT G-3′ (Hind III site), and for GlcATs: sense: 5′-AAA AGC TTT ACC TCA ATT TTC AGT GTG T-3′ (Xho-1 site) antisense: 5′-AAC TCG AGA TGA AGT CCG CGC TTT TCA C-3′ (Hind III site). The EGFP-GlcATp/GlcATs vector was obtained by ligation of an Xho-1/Hind III fragment from pcDNA3.1-GlcATp/GlcATs into EGFP-N1. Transfection was performed using electroporation as described previously (≡1 μg of plasmid/1×106 cells). A group of cells was transfected with an equivalent amount of combined EGFP-GlcATp and EGFP-GlcATs plasmid (0.5 μg each). The rate of success of the procedure was observed by the expression of GFP in transfected cells after 24 h of incubation.

### Results

To gain further insight into the precise role of SGPG in endothelial cell functions, a gain-of-function experiment was performed by upregulating the SGPG expression level by inflammatory cytokines. cDNAs of GlcATp and GlcATs were cloned, and the cells were transfected with EGFP-GlcATp, EGFP-GlcATs, and the combined clones. To identify the efficacy of GlcATp and GlcATs transfection, the transfected cells were visualized under a fluorescent microscope for GFP expression and then identified GFP expression by Western blot analysis using GFP-antibody. One single protein band was detected in GFP transfection, two protein bands were detected in GlcATp/GlcATs-GFP transfection, and three protein bands were detected in the combined transfection experiment (results not shown). In addition, the SGPG concentrations in all transfected cells were measured to verify that SGPG expression was indeed stimulated.

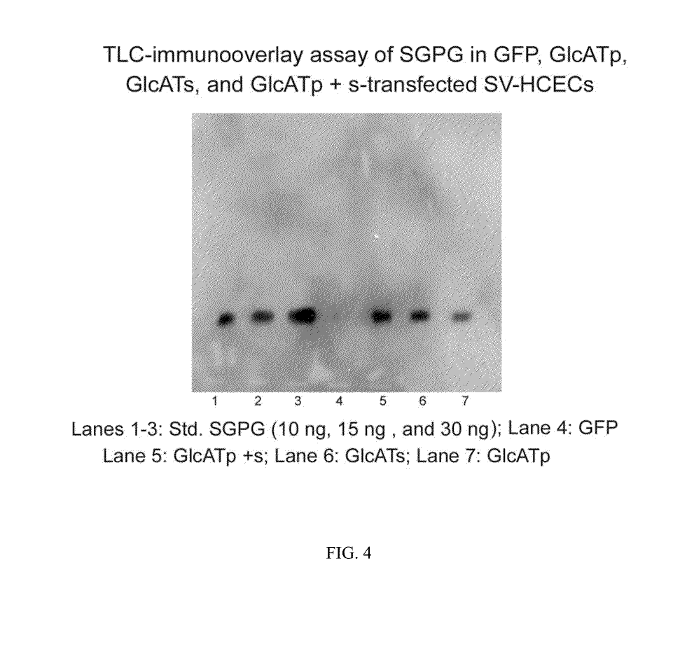

Briefly, the transfected cells were cultured for a defined period of incubation (24-48 h), and the cells were collected after mild trypsinization. SGPG concentration was measured using the purified lipid fraction in the lipid extract employing MAb NGR50 in a TLC-immuno-overlay method (Dasgupta S, et al., J Neurosci Res., 85:1086-1094 (2007)). It was discovered that the level of SGPG was enhanced by GlcATp and GlcATs transfection, and the efficacy of upregulation was GlcATp+GlcATs>GlcATs>GlcATp, corresponding to a 20-, 12-, and 8-fold increase (FIG. 4), respectively, in SGPG concentration compared to the control (EGFP).

### Example 3

### GlcATp/GlcATs-Transfection Reduced NF-κB Activity by Inhibiting IKB Activation

### Materials and Methods

NF-κB Activity Assay

Along with the EGFP-GlcATp and EGFP-GlcATs plasmids, cells were co-transfected using 0.8 μg of pNF-κB luciferase, a multimerized κB-luciferase reporter gene plasmid, and 0.4 μg of pRL-CMV (Renilla luciferase) internal control plasmid to normalize the efficacy of the transfection procedure. After 24 h of incubation, cell lysate was prepared and the level of luciferase activity was determined using the dual luciferase reporter system in accordance with the instruction of the manufacturer (Promega, Madison, Wis.).

### Results

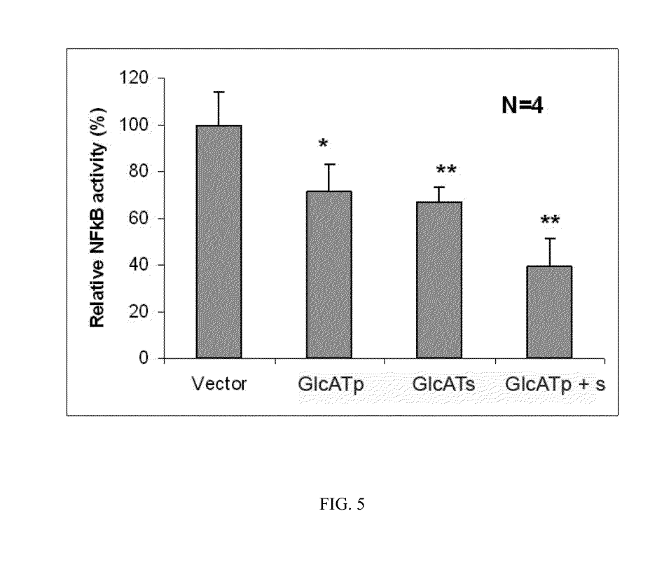

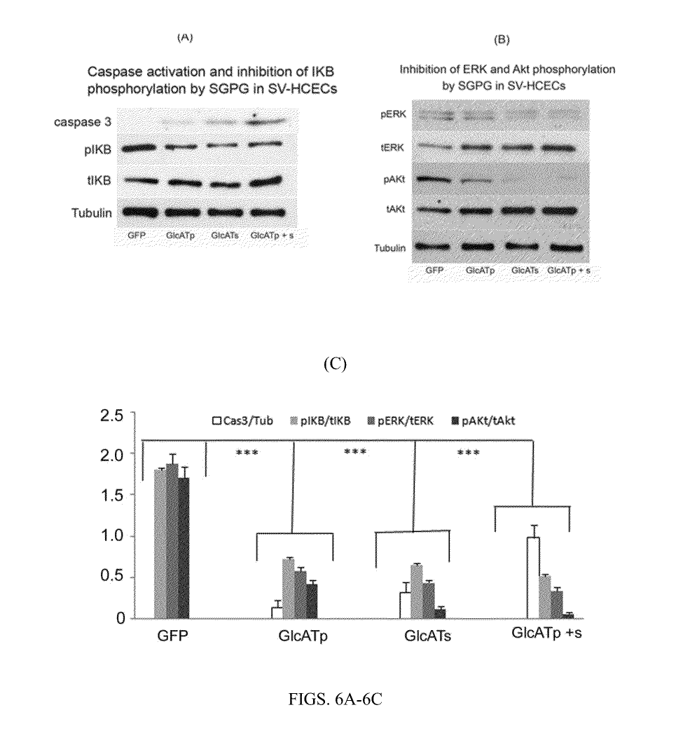

A luciferase assay was used to determine the effect of SGPG expression on NF-κB activity in the transfected cells. The empty vector (EGFP—N1) was used as a control. The GlcATp and GlcATs transfected cells showed reduced NF-κB activity compared to that of the control cells (FIG. 5), and that reduction is further supported by the inhibition of IKB activation (FIG. 6A) as shown by Western blot analysis. SGPG inhibition also reduced NF-κB activity in HNK-1ST siRNA transfected cells, which down-regulated SGPG expression after cytokine stimulation (Dasgupta S, et al., J Neurosci Res., 87:3591-3599 (2009)).

### Example 4

### GlcATp/GlcATs-Transfection Promotes Cell Apoptosis

Since NF-κB is involved in cell survival pathways, the apoptosis level in the GlcATp- and GlcATs-transfected cells were examined by measuring caspase 3 activity. FIG. 6A shows that caspase 3 was activated in GlcATp- and GlcATs-transfected cells, for which GlcATs exhibited a stronger effect than GlcATp. The double transfection (GlcATp+GlcATs), however, shows a strong synergistic effect compared to GlcATp or GlcATs transfection alone (FIG. 5A). Taken together, the data indicate that increased SGPG expression induced cell apoptosis in SV-HCECs. To understand the apoptotic mechanism relevant to cell survival signals, the PI3 kinase (Akt) and MAP kinase (ERK) activation were determined using Western blot analysis and found that the transfected cells showed a downregulation of phospho-Akt and phospho-ERK activity (FIG. 6B).

### Example 5

### Modulating SGPG Expression Inhibits NF-κB Activity Through TNFα-Receptor Signaling

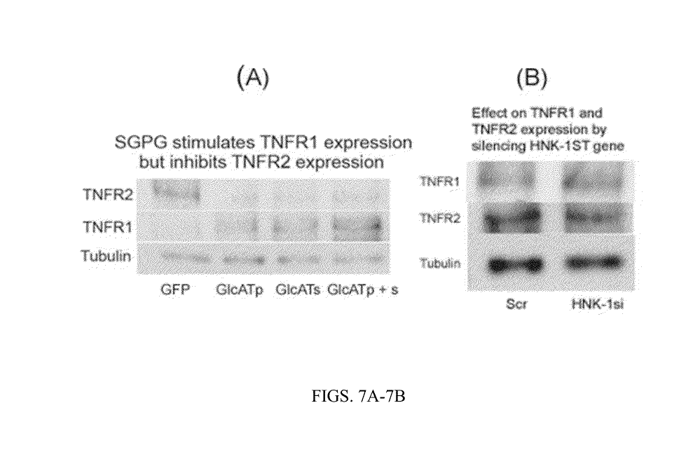

To further investigate SGPG regulation in inhibiting NF-κB activity, TNFα-receptor (TNFR1 and TNFR2) expression was examined after HNK-1ST gene silencing and in SGPG elevation by GlcATp/GlcATs transfected cells as these receptors are involved in regulation of NF-κB activity and are associated with both cell death and cell survival pathways, respectively (McCoy M K, et al., J Neuroinflammation., 5:45 (2008)). The data indicated that silencing SGPG expression activated TNFR2 expression, and that elevation of SGPG expression stimulated TNFR1 and reduced TNFR2 expression (FIGS. 7A and 7B).

### Example 6

### Further Determination of Cell Apoptosis Induced by SGPG

### Materials and Methods

Immuno-Overlay Analysis of the SGPG Concentration in Transfected Cells

Since SGPG is a minor component of the total GSLs in SV-HCEC (Muruganandam A, et al., FASEB J., 11:1187-1197 (1997); Duvar S, et al., J. Neurochem., 75:1970-1976 (2000)), the control (EGFP-transfection) and transfected cells were cultured separately in 150-mm dishes to obtain a sufficient number of cells for SGPG analysis by an immuno-overlay method. Control (EGFP-transfected) cells and EGFP-GlcATp/GlcATstransfected cells were grown for 48 h as described. Cells were then collected, washed with cold PBS, and preserved at −20° C. before use. Lipids were extracted from cells using solvent mixtures (chloroform:methanol:water 2:4:1, v/v; followed by chloroform:methanol 2:1, v/v), and the SGPG fraction was purified from the lipid extract using DEAE A-25 (acetate form) as described previously (Dasgupta S, et al., J. Neurosci. Res., 85:1086-1094 (2007)). Each purified fraction was dissolved in a defined volume of solvent (determined by the protein content) and an equal volume of the samples was applied to an HPTLC with standard SGPG. The plate was developed using the solvent system of chloroform:methanol:0.25% CaCl₂ (55:45:10; v/v), coated, and exposed to MAb NGR 50 (specific for SGPG), followed by a mouse peroxidase conjugated-secondary antibody. After washing with PBS, a chemiluminescence reagent, ECL, was added to the plate, and the bands were revealed by exposing to an X-ray film (Dasgupta S, et al., J Neurosci Res., 85:1086-1094 (2007)).

Fluorochrome Inhibitor of Caspases Assay (FLICA)

Using FLICA, cell death induced by SGPG expression was determined. Staining of active caspases using the FLICA assay was performed with EGFP-GlcAT-plasmids transfected SV-HCECs using sulforhodamine-labeled fluoromethyl ketone peptide inhibitor (red) according to the manufacturer's instructions (Immunochemistry Technologies). Cells were transfected with the respective plasmid, grown on a cover-slip for 24-48 h, and then stained for active caspases using FLICA. Briefly, the FLICA reagent was added to the medium and the cells incubated for 1 h at 37° C. under 5% CO2. The cells were washed once with washing solution and then fixed with 4% p-formaldehyde (PFA) in phosphate-buffered saline (PBS) for further analysis.

Immunocytochemical Localization of SGPG and Caspase 3

To verify the effect of GlcATp/GlcATs transfection on SGPG expression, the localization of SGPG in control and GlcATp/GlcATstransfected cells were probed immunocytochemically (Dasgupta S, et al., J Neurosci Res., 85:1086-1094 (2007)). Cells were cultured on cover slips and grown 24-48 h, washed with 1× Hank's balanced salt solution, and fixed using 4% PFA. The fixed cells were permeabilized and then treated with MAb NGR 50 and caspase 3 antibody, followed by an appropriate secondary antibody (anti-mouse IgG or anti-rabbit IgG) conjugated with cy3 or cy5. Cells were further stained with Hoechst 33258 (nuclear stain) and visualized under a confocal microscope.

Statistical Evaluation

Data are expressed as means±standard deviations (SD) from 3-5 independent experiments. Statistical significance was determined using Student's t-test for comparison between two means and by two way ANOVA in MS EXCEL v2007.

### Results

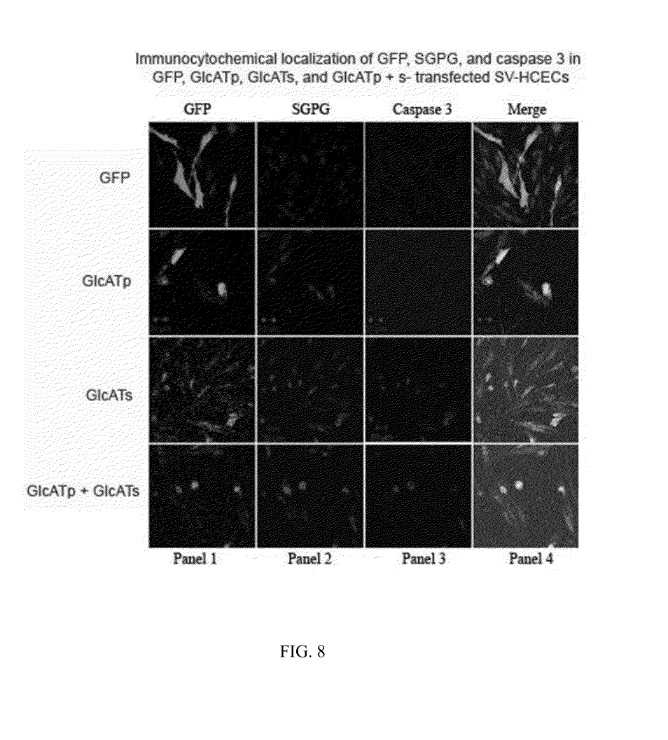

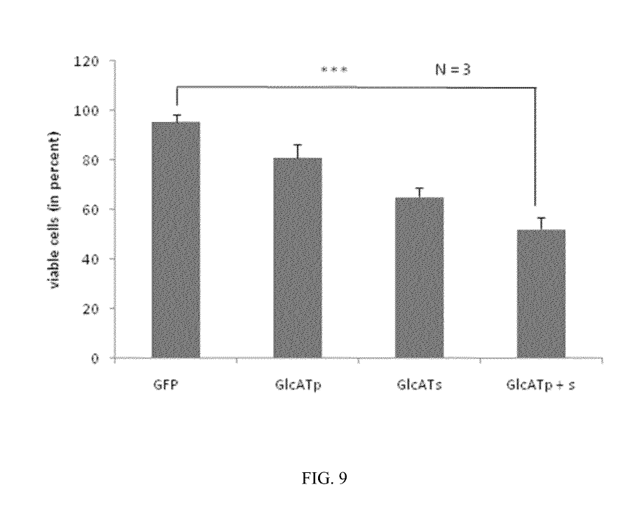

To further evaluate the effect of SGPG expression on cell death, immunofluorescence and FLICA were performed. Immunofluorescence of SGPG and activated caspase 3 showed that cells which expressed EGFP-GlcATp (green), EGFP-GlcATs (green), or both, had higher SGPG expression (red) and stained positive for active caspase 3 (FIG. 8), which confirms the lipid measurement and Western blot analysis data. Using FLICA, it was discovered found that GlcATp and GlcATs transfection promoted cell death by 15-20% and 35-40%, respectively, while reaching approximately 50% or more cell death in cells transfected with a combination of GlcATp and GlcATs (FIG. 9).

By regulating the expression of SGPG (gain-of-function and loss-of function studies), a novel role of SGPG in cell apoptosis was established. Since down-regulation for SGPG expression by HNK-1ST siRNA reduced the NF-κB activity, overexpression of SGPG was predicted to stimulate such activity. It was determined that NF-κB activity was also inhibited in both GlcATp and GlcATs-transfected cells, and the efficacy of inhibition is GlcATp<GlcATs<GlcATp+GlcATs. The results of NF-κB inhibition were further confirmed by inhibition of IKB phosphorylation. These observations further underscore the complexity of NF-κB activation, as has been documented in the literature (Moscat J, et al., Nat. Immunol., 12:12-14 (2011)). Thus, the finding raises an important issue regarding the precise mechanism for NF-κB activity regulation relevant to SGPG expression. To delineate the mechanism of NF-κB inhibition by silencing SGPG (by HNK-1ST siRNA) expression, TNFα-receptors 1 and 2 (TNFR1 and TNFR2) expression was examined employing Western blot analysis. Transfection of siRNA led to the reduction of TNFR1, but showed no effect on TNFR2 (FIG. 7B). SGPG over-expression by GlcATp/GlcATs transfection, however, resulted in a reduction of the level of TNFR2 and an elevation of the level of TNFR1 (FIG. 7A). TNFR1 is expressed in most cell types and can be activated by binding of either soluble or trans-membrane TNF, with a preference for soluble TNF. By contrast, TNFR2 is expressed primarily by microglia and endothelial cells and is preferentially activated by trans-membrane TNF (McCoy M K, et al., J. Neuroinflammation., 5:45 (2008)). Elevation of soluble TNF is a hallmark of conditions of certain chronic neuro-inflammation, including multiple sclerosis, amyotrophic lateral sclerosis, and Parkinson's disease (McCoy M K, et al., J Neuroinflammation., 5:45 (2008)).

TNFR1 signaling has been reported to stimulate cell apoptosis via complex II in NF-κB mediated signaling (Micheau O, et al., Cell., 114:181-190 (2003)). TNFR1 inhibition, by silencing the HNK-1^{ST} gene, is consistent with down-regulation of caspase 3 activity by HINK-1^{ST} siRNA transfection with reduction of NF-κB activity (pro-apoptotic). Signaling through TNFR2 activates inflammatory and pro-survival signaling pathways through recruitment of TRAF1 and TRAF2 adaptor proteins and subsequent activation of the NF-κB pathway (Rothe M, et al., Cell., 83:1243-1252 (1995); McCoy M K, et al., J. Neuroinflammation., 5:45 (2008); Rothe M, et al., Cell., 78:681-692 (1994); Rao P, et al., J. Interferon Cytokine Res., 15:171-177 (1995)). TNFR2 does not contain a death domain and, thus, unlike signaling through TNFR1, TNFR2 activation does not lead to caspase activation (McCoy M K, et al., J. Neuroinflammation., 5:45 (2008)). Overall, TNFR2 activation is believed to initiate primarily pro-inflammatory and pro-survival signaling (McCoy M K, et al., J. Neuroinflammation., 5:45 (2008)). The data indicate that SGPG expression mediates cell apoptosis by inhibiting TNFR2 and by stimulating TNFR1 expression and caspase 3-activation (cell death signaling). This activity is reflected by a reduction of NF-κB activity (cell survival); by contrast, SGPG inhibition by HNK-1ST reduces NF-κB activity (apoptotic signal) by inhibiting the TNFR1 expression that leads to cell survival.

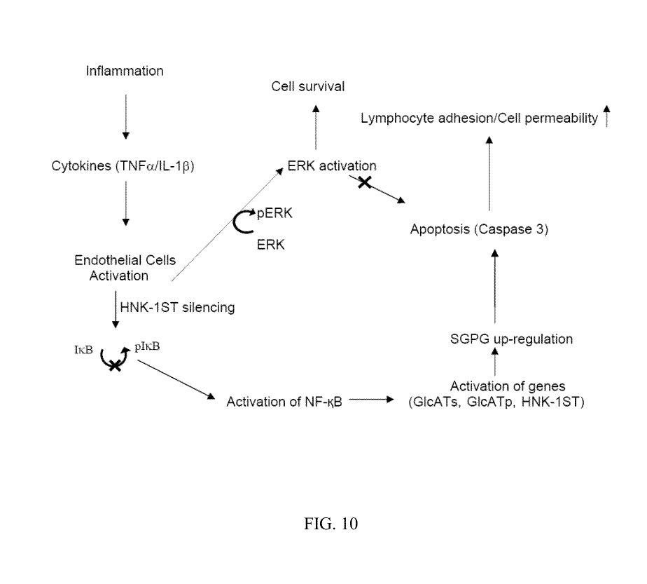

The GlcATp/GlcATs-transfected cells apparently showed enhanced cell death; the cultures were found to contain fewer viable cells with the increasing time of incubation. More viable cells were observed after 24 h of incubation as compared to 48 h of incubation, and this observation was confirmed by FLICA assay. To correlate apoptosis and SGPG expression, TLC-immunooverlay assay was used to quantitate SGPG concentration. Additionally, SGPG regulation was examined by immuno-cytochemistry along with GFP expression and caspase 3 activity assays. SGPG concentration was up-regulated in the transfected cells. The order of expression level was GlcATp<GlcATs<GlcATp+GlcATs, a similar profile to that observed in caspase 3 activation as well as in NF-κB inhibition. It is noteworthy that active caspase 3 expression was elevated specifically in cells that showed a higher level of SGPG expression (also identified by GFP expression), while cells with only GFP (control) transfection showed neither SGPG over-expression nor cell death (FIG. 8). After 48 h of incubation, approximately 45%-50% or more of cell death was observed in cells transfected with combined EGFP-GlcATs and EGFP-GlcATp, while transfection with either one, independently prompted 30%-35% and 15%-20% cell death, respectively. Western blot analysis of the cell lysate indicated that caspase 3 was activated to a large extent, although the other survival pathways, Akt and ERK, were inhibited by SGPG overexpression. Again, the number of cell death was proportional to the degree of NF-κB inhibition. In addition, it has been demonstrated that GlcATs is the predominant gene in SV-HCECs; its expression was more highly stimulated by cytokines than by GlcATp (Dasgupta S, et al., J Neurosci Res., 85:1086-1094 (2007)). The present study further extended that observation and showed that over-expression of GlcATs had a detrimental effect on cell viability. Hence, it has been unequivocally established that in addition to SGPG's other cellular functions, its expression under inflammatory conditions is a death signal for endothelial cells, and an inhibition of their expression prevents T cell adhesion and protects against cell death (FIG. 10).

Evidence indicates that T cells routinely survey the BBB by infiltrating the barrier under normal conditions to maintain homeostasis of the nervous system (Hickey W F, Glia., 36:118-124 (2001)) along with their apparent ability to repair the nervous system (Schwartz M, et al., Immunol. Today, 21:265-268 (2000); Hohlfeld R, et al., J. Neuroimmunol., 107:161-166 (2000)). However, the endothelial cell death may affect the integrity of BBB/BNB and increase cellular permeability by loosening tight junctions, as involvement of endothelial cell death/dysfunction has been implicated in pathogenesis of many neurological disorders such as stroke, focal cerebral ischemia, and Alzheimer disease (Nagasawa H, et al., Stroke, 20:1037-1043 (1989); Zipfel G J, et al., Stroke, 40:S16-19 (2009); Deininger M H, et al., J. Neurosci., 22:10621-10626 (2002); Cheng Y D, et al., NeuroRx., 1:36-45 (2004); Jimenez B, et al., Nat. Med., 6:41-48 (2000); Hossmann K A, Ann. Neurol., 36:557-65 (1994); Mahad D J, et al., Mult Scler., 9:189-198 (2003)). Endothelial cell apoptosis may cause the breakdown of the barrier, leading to vasogenic edema (Rizzo M T, et al., Mol. Neurobiol., 42:52-63 (2010)). In addition, the elimination or prevention of endothelial cell dysfunction and death is critical for tissue homeostasis (Wyllie A H, et al., Int. Rev. Cytol., 68:251-306 (1980)) and, due to inappropriate regulation, apoptosis can also promote, contribute to, and even exacerbate the disease process (Hetts S W, JAMA., 279:300-307 (1998)). Hence, elucidating the mechanism of endothelial cell death and/or dysfunction to disease processes could help in advancing the knowledge and in developing new therapies for certain neurological disorders (Rizzo M T, et al., Mol. Neurobiol., 42:52-63 (2010)). Although brain microvascular endothelial cells were used, a similar mechanism is also thought to apply to BNB, as endothelial cells are also integral component of the BNB in maintaining the barrier function, which is critical in peripheral neuropathological conditions, such as GBS.

In an in vivo model, an inflammatory signal proceeds via infiltration of T cells, phagocytic cells, cytokines, and chemokines through the BBB/BNB. These cells and macromolecules then gain access to the nerve tissues, initiating the cell-mediated degenerative process. At any stage, an autoimmune response can also be triggered by an auto-antibody and malfunction of the immune system. This auto-antibody can easily penetrate a damaged BBB/BNB barrier, leading to destruction of the CNS/PNS and propagating the disease progression. Hence, the passage of the invading molecules through the BBB/BNB is one of the most important criteria for the onset and development of the degenerative process. A classical example of such failure of auto-antibody penetration has already been documented indicating that a very high titer auto-antibody raised in rabbits did not initiate any CNS/PNS demyelination (Dasgupta S, et al., Neurochem. Res., 29:2147-2152 (2004)). Hence, it is concluded that SGPG concentration in endothelial cells can regulate the attachment and penetration of activated T cells and phagocytes under inflammation, and in maintaining normal barrier function. The data herein indicate that inhibition of SGPG expression can be a viable strategy for designing a suitable in vivo cell-permeability inhibitor. Such an inhibitor can be used as a potential therapeutic agent in neuro-inflammatory diseases by preventing endothelial cell death and protecting the nervous system from invasion by circulating immune cells, pathogenic immunoglobulins, or other bio-degrading macromolecules.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method for maintaining integrity of the blood-brain barrier in a subject comprising administering to the subject a glucuronosyltransferase antagonist, an antagonist of killer epitope-1 sulfotransferase (HNK-1ST), or a combination thereof, in an amount effective to reduce expression of sulfated glucuronosyl paragloboside (SGPG) in the subject and thereby reduce apoptosis of endothelial cells of the blood-brain barrier or the blood-nerve barrier in the subject.

2. The method of claim 1, wherein the glucuronosyltransferase antagonist is selected from the group consisting of siRNA and antisense nucleic acids specific for nucleic acids encoding the glucuronosyltransferase.

3. The method of claim 2, wherein the glucuronosyltransferase is selected from the group consisting of GlcATp and GlcATs.

4. The method of claim 1, wherein the antagonist of HNK-1ST is selected from the group consisting of siRNA or antisense nucleic acids specific for nucleic acids encoding HNK-1ST.

5. A method of treating neuro-inflammatory disease in a subject in need thereof comprising administering to the subject a glucuronosyltransferase antagonist, an antagonist of HNK-1ST, or a combination thereof, in an amount effective to reduce expression of SGPG in the subject and thereby reduce apoptosis of endothelial cells and thereby reduce invasion of the subject's nervous system by immune cells, pathogenic immunoglobins, bio-degrading molecules, or combinations thereof.

6. The method of claim 5, wherein the glucuronosyltransferase antagonist is selected from the group consisting of siRNA and antisense nucleic acids specific for nucleic acids encoding the glucuronosyltransferase.

7. The method of claim 6, wherein the glucuronoslytransferase is selected from the group consisting of GlcATp and GlcATs.

8. The method of claim 5, wherein the antagonist of HNK-1ST is selected from the group consisting of siRNA or antisense nucleic acids specific for nucleic acids encoding HNK-1ST.

9. A method for reducing cytokine-induced cell permeability of endothelial cells comprising administering to the endothelial cell an effective amount of a glucuronosyltransferase antagonist, an antagonist of killer epitope-1 sulfotransferase (HNK-1ST), or a combination thereof, to reduce expression of sulfated glucuronosyl paragloboside (SGPG) in the subject and thereby reduce cytokine-induced cell permeability of the endothelial cell.

10. The method of claim 9, wherein the glucuronosyltransferase antagonist is selected from the group consisting of siRNA and antisense nucleic acids specific for nucleic acids encoding the glucuronoslytransferase.

11. The method of claim 10, wherein the glucuronoslytransferase is selected from the group consisting of GlcATp and GlcATs.

12. The method of claim 5, wherein the antagonist of HNK-1ST is selected from the group consisting of siRNA or antisense nucleic acids specific for nucleic acids encoding HNK-1ST.
